SEQUENCE LISTING (5 sequences)

SEQ ID NO: 1 - RNA, 21 nt, Homo sapiens
uaaagugcugacagugcagau

SEQ ID NO: 2 - RNA, 23 nt, Homo sapiens
caaagugcuguucgugcagguag

SEQ ID NO: 3 - RNA, 22 nt, Homo sapiens
cauugcacuugucucggucuga

SEQ ID NO: 4 - DNA, 855 bp, Homo sapiens
  CDS (1)..(852)
atgtcgatgctgccgtcgtttggctttacgcaggagcaagtggcgtgcgtgtgcgaggttctgcagcaaggcggaaacctggagcgcctgggcaggttcctgtggtcactgcccgcctgcgaccacctgcacaagaacgagagcgtactcaaggccaaggcggtggtcgccttccaccgcggcaacttccgtgagctctacaagatcctggagagccaccagttctcgcctcacaaccaccccaaactgcagcaactgtggctgaaggcgcattacgtggaggccgagaagctgcgcggccgacccctgggcgccgtgggcaaatatcgggtgcgccgaaaatttccactgccgcgcaccatctgggacggcgaggagaccagctactgcttcaaggagaagtcgaggggtgtcctgcgggagtggtacgcgcacaatccctacccatcgccgcgtgagaagcgggagctggccgaggccaccggcctcaccaccacccaggtcagcaactggtttaagaaccggaggcaaagagaccgggccgcggaggccaaggaaagggagaacaccgaaaacaataactcctcctccaacaagcagaaccaactctctcctctggaagggggcaagccgctcatgtccagctcagaagaggaattctcacctccccaaagtccagaccagaactcggtccttctgctgcagggcaatatgggccacgccaggagctcaaactattctctcccgggcttaacagcctcgcagcccagtcacggcctgcagacccaccagcatcagctccaagactctctgctcggccccctcacctccagtctggtggacttggggtcctaa

SEQ ID NO: 5 - protein, 284 aa, Homo sapiens
MSMLPSFGFTQEQVACVCEVLQQGGNLERLGRFLWSLPACDHLHKNESVLKAKAVVAFHRGNFRELYKILESHQFSPHNHPKLQQLWLKAHYVEAEKLRGRPLGAVGKYRVRRKFPLPRTIWDGEETSYCFKEKSRGVLREWYAHNPYPSPREKRELAEATGLTTTQVSNWFKNRRQRDRAAEAKERENTENNNSSSNKQNQLSPLEGGKPLMSSSEEEFSPPQSPDQNSVLLLQGNMGHARSSNYSLPGLTASQPSHGLQTHQHQLQDSLLGPLTSSLVDLGS

(19) United States
(12) Patent Application Publication
(10) Pub. No.: US 2013/0243787 A1
(43) Pub. Date: Sep. 19, 2013

(54) Predicting TGF-beta Therapeutic Responses

(71) Applicants: Heide Ford, Aurora, CO (US); Anna Smith, Aurora, CO (US)

(72) Inventors: Heide Ford, Aurora, CO (US); Anna Smith, Aurora, CO (US)

(21) Appl. No.: 13/605,736

(22) Filed: Sep. 6, 2012

Related U.S. Application Data
(60) Provisional application No. 61/531,965, filed on Sep. 7, 2011.

(51) Int. Cl. C12Q 1/68

(52) U.S. Cl. CPC C12Q 1/6881
USPC 424/158.1; 506/9

(57) ABSTRACT

The invention provides methods of determining whether a cancer in a subject is responsive to anti-TGF-β therapy. In some embodiments, the invention further provides for the administration of an anti-TGF-β therapy cancer therapy to the subject.

This application claims benefit of priority to U.S. Provisional Application Ser. No. 61/531,965, field Sep. 7, 2011, the entire contents of which are hereby incorporated by reference.

This invention was made with government support under grant nos. ROI-CA095277 and W81XWH-10-1-0296 by the National Institutes of Health and the Department of Defense. The government has certain rights in the invention.

### BACKGROUND OF THE INVENTION

I. Field of the Invention

The present invention relates generally to the fields of oncology, molecular biology, and medicine. More particularly, the invention relates to the use of monitoring of miRNA expression as an indicator of therapeutic efficacy for anti-TGF-β cancer treatments.

II. Description of Related Art

Cancer shares many common properties with normal development. During normal development of an organ, genes are activated to stimulate the proliferation and survival of progenitor cells, as well as to stimulate migration, invasion, and neovascularization. These genes are usually down-regulated once organ development is completed. In cancer, the same genes are often re-activated, stimulating inappropriate proliferation, survival, migration, invasion, and neovascularization. Thus, there is a need for treatments that inhibit the expression of such genes after organ development is completed. One such example is transforming growth factor beta (TGF-β).

TGF-β is a secreted protein that controls proliferation, cellular differentiation, and other functions in most cells. It plays a role in immunity, cancer, heart disease, diabetes, Marfan syndrome and Loeys-Dietz syndrome. TGF-β exists in multiple isoforms called TGF-β1, TGF-β2 and TGF-β3. The TGF-β family is part of a superfamily of proteins known as the transforming growth factor beta superfamily, which includes inhibins, activin, anti-müllerian hormone, bone morphogenetic protein, decapentaplegic and Vg-1. Importantly, TGF-β acts as an anti-proliferative factor in normal epithelial cells and at early stages of oncogenesis, but becomes tumor-promoting in later stages due to cancer cells inducing autocrine signaling by increasing their production of both TβR1 and TGF-β, the latter of which also acts on surrounding cells.

The importance of perturbation in TGF-β signaling for the onset and progression of cancer is well-established. Many tumors overexpress TGF-β, and high circulating levels of TGF-β1 in cancer patients are frequently associated with poor prognosis. TGF-β has context-dependent biphasic action during tumorigenesis. Because of this, it is essential to take due care about the selection of patients that will benefit from anti-TGF-β therapy, to avoid treating patients that will not respond, and even further, to avoid harming those patients in which TGF-β is tumor-preventive. Methods permitting the clinician to distinguish among these patients would be a major advance in the field of cancer therapy.

### SUMMARY OF THE INVENTION

In one aspect, the invention provides a method of predicting response of a cancer patient to an anti-TGF-β therapy comprising (a) obtaining expression information on one or more miR's from the miR 106b-25 cluster in a patient sample that contains miR's from the miR 106b-25 cluster; and (b) classifying said subject as (i) anti-TGF-β-responsive if one or more miR's are observed to be upregulated as compared to normal tissue; or (ii) anti-TGF-β-non-responsive if one or more miR's are not observed to be upregulated as compared to normal tissue. The method may further comprise making a treatment decision for said patient. The method may further comprise measuring miR expression levels for one or more miR's that are upregulated by Six-1. The method may further comprise obtaining said sample, such as a tumor sample. The outcome may be that none, one, two or all three of miR-106b, miR-93 and miR-25 are elevated.

The treatment decision may be not to treat said patient with an anti-TGF-β therapy, and may further comprise treating said patient with a therapy other than anti-TGF-β. The treatment decision may be to treat said patient with an anti-TGF-β therapy, and may further comprise treating said patient with an anti-TGF-β therapy. The anti-TGF-β therapy may be an anti-TGF-β antibody or a anti-TGF-β small molecule. The method may further comprise performing steps (a) and (b) a second time, such as where the second time follows an anti-TGF-β therapy.

The cancer patient may be a human patient. The cancer patient may have breast cancer, Wilm's tumor, ovarian cancer, adenocarcinoma, adenosquamous carcinoma, papillary carcinoma, secretory carcinoma, sarcomatoid carcinoma, hepatocellular carcinoma, osteosarcoma, rhabdomyosarcoma or a peripheral nerve sheath tumor. The cancer patient may have an epithelial cell cancer. The cancer patient may have metastatic, multi-drug resistant or recurrent cancer.

In another embodiment, there is provided a method of assessing TβRI expression upregulation in a cancer tissue sample comprising (a) obtaining expression information on one or more miR's from the miR 106b-25 cluster from said sample; and (b) identifying TβRI upregulation if one or more miR's are observed to be upregulated as compared to normal tissue.

In still another embodiment, there is provided a method of assessing increased TGF-β signaling in a cancer tissue sample comprising (a) obtaining expression information on one or more miR's from the miR 106b-25 cluster from said sample; and (b) identifying increased TGF-β signaling if one or more miR's are observed to be upregulated as compared to normal tissue.

In yet another embodiment, there is provided a method of predicting response of a cancer patient to an anti-TGF-β therapy comprising (a) obtaining expression information on Six-1 in a patient sample; and (b) classifying said subject as (i) anti-TGF-β-responsive if Six-1 is observed to be upregulated as compared to normal tissue; or (ii) anti-TGF-β-non-responsive if Six-1 is not observed to be upregulated as compared to normal tissue. The method may further comprise making a treatment decision for said patient. The method may further comprise measuring Six-1 expression levels. The method may further comprise obtaining said sample, such as a tumor sample.

The treatment decision may be to treat said patient with an anti-TGF-β therapy, and may further comprise treating said patient with an anti-TGF-β therapy. The anti-TGF-β therapy may be an anti-TGF-β antibody or a anti-TGF-β small molecule. The method may further comprise performing steps (a) and (b) a second time, such as where the second time follows an anti-TGF-β therapy.

The cancer patient may be a human patient. The cancer patient may have breast cancer, Wilm's tumor, ovarian cancer, adenocarcinoma, adenosquamous carcinoma, papillary carcinoma, secretory carcinoma, sarcomatoid carcinoma, hepatocellular carcinoma, osteosarcoma, rhabdomyosarcoma or a peripheral nerve sheath tumor. The cancer patient may have an epithelial cell cancer. The cancer patient may have metastatic, multi-drug resistant or recurrent cancer.

In still yet another embodiment, there is provided a method of assessing TβRI expression upregulation in a cancer tissue sample comprising (a) obtaining expression information on Six-1 from said sample; and (b) identifying TβRI upregulation if Six-1 is observed to be upregulated as compared to normal tissue.

In an additional embodiment, there is provided a method of assessing increased TGF-β signaling in a cancer tissue sample comprising (a) obtaining expression information on Six-1 from said sample; and (b) identifying increased TGF-β signaling if Six-1 is observed to be upregulated as compared to normal tissue.

Any embodiment discussed with respect to one aspect of the invention applies to other aspects of the invention as well.

The embodiments in the Example section are understood to be embodiments of the invention that are applicable to all aspects of the invention.

The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Following long-standing patent law, the words “a” and “an,” when used in conjunction with the word “comprising” in the claims or specification, denotes one or more, unless specifically noted.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these figures in combination with the detailed description of specific embodiments presented herein.

FIGS. 1A-B. A miRNA microarray identifies the miR-106b-25 cluster family members as upregulated by Six1. (FIG. 1A) miRNAs that are significantly up- or downregulated (P value <0.05) in MCF7-Six1 vs. MCF7-Ctrl cells as determined by a miRNA profiling array (FIG. 1B) Schematic representation of the miR-106b-25 cluster of miRNA (miR-106b, miR-93, and miR-25) within the 13th intron of the MCM7 gene.

FIGS. 2A-C. Six1 regulates the miR-106b-25 Cluster. (FIG. 2A) Stable overexpresson of Six1 in MCF7 cells leads to an increase in miR-106b, miR-93, and miR-25 as determined using qRT-PCR. Data are represented as the mean+/−SEM of three individual MCF7-Six1 and MCF7-Ctrl clones. (FIG. 2B) Knockdown of Six1 in 21PT cells using Six1 specific siRNA (siSix1, 50 nm and 100 nm) leads to a decrease in expression of all 3 miRNA in the miR-106b-25 Cluster when compared to a control knockdown (siNeg). For qRT-PCR analysis, the average of 3 replicates +/−SD is shown. (FIG. 2C) RNA was isolated from the mammary glands of bitransgenic mice in which Six1 was induced with doxycycline (TO-Six1+Dox) versus single transgenic MTB control mice also treated with Dox (MTB+Dox), but unable to express Six1. qRT-PCR performed on the isolated RNA for the miR-106b-25 miRNAs demonstrates an increase in expression of all three miRNAs in the TO-Six1+Dox mammary glands, which express high levels of the Six1 transgene as compared to MTB+Dox control mammary glands. n=3 mice for each condition, and each miRNA was normalized to U6 RNA. P values represent statistical analysis using a paired t test.

FIGS. 3A-D. The miR-106b-25 miRNAs repress Smad7. (FIG. 3A) qRT-PCR reveals a decrease in Smad7 mRNA in MCF7-Six1 cells versus MCF7-Ctrl cells. Data shown are the result of 3 replicate qRT-PCR reactions (+/−SD), normalized to cyclophilin B mRNA levels. (FIG. 3B) A Renilla luciferase reporter containing the 3′UTR of Smad7 was transfected into MCF7 cells containing an empty vector (MCF7-EV), a non-silencing control (MCF7-NS), or the miR-106b-25 cluster (MCF7-Cluster). Measurement of renilla luciferase normalized to firefly luciferase (present on the same vector but expressed from a different promoter) demonstrates a significant repression of the Smad7 3′UTR in response to miR-106b-25 expression. (FIG. 3C) Western blot analysis demonstrates that MCF7-Cluster cells have decreased Smad7 protein as compared to MCF7-EV and MCF7-NS cells. (FIG. 7D) MCF7-Ctrl and MCF7-Six1 cells treated with miRNA inhibitors towards miR-106b, and miR-93, show a de-repression of Smad7 protein in MCF7-Six1 cells. P values represent statistical analysis using a paired t test.

FIGS. 4A-E. The miR-106b-25 cluster activates TGF-β signaling. (FIG. 4A) Transient and (FIG. 4B) stable overexpression of the miR-106b-25 cluster in MCF7 cells leads to increased expression of TβRI protein over controls and an increase in phosphorylated Smad3 (p-Smad3). (FIG. 4C) MCF7-Ctrl and MCF7-Six1 cells expressing stable miRZip inhibitors targeting the miR-106b-25 miRNAs individually and together (miRZip-Cluster) show a reversal of the Six1-induced increase in TβRI protein in miRzip-93 and miRZip-Cluster treated MCF7-Six1 cells as compared to scramble miRZip controls (miRZip-SCR). (FIG. 4D) Introduction of miRZip-106b, miRZip-93, and miRZip-Cluster into MCF7-Six1 cells reverses the Six1-induced increase in p-Smad3 levels, without affecting the Six1-induced increase in total Smad3 levels. (FIG. 4E) Areal-time PCR array containing TGF-β transcriptional targets shows enrichment for TGF-β target gene expression in MCF7-Cluster cells over MCF7-NS cells. Data is represented as-fold change expression of MCF7-Cluster compared to control MCF7-NS from 3 replicate plates of each condition.

FIGS. 5A-D. The miR-106b-25 cluster mediates features of EMT. (FIG. 5A) miR-106b-25 overexpression results in loss of E-cadherin and β-catenin from the insoluble (cytoskeleton-associated) protein fraction of the cell as determined by western blot analysis (left). E-cadherin and β-catenin levels were quantified and are graphed as the ratio of insoluble to soluble (cytosolic) protein (right). (FIG. 5B) MCF7-Cluster cells show increased activity of the β-catenin responsive-luciferase reporter TOP-Flash, normalized to Renilla luciferase activity. Data are from 3 replicates +/−SD. (FIG. 5C) The Six1-induced increase in TOP-Flash activity is reversed with inhibition of the miR-106b-25 cluster using transient hairpin inhibitors (Dharmacon, miRidian). (FIG. 5D) Expression of the miR-106b-25 Cluster in MCF7 cells results in decreased adhesion to cell matrix proteins Collagen I, Collagen IV, and Fibronection, similar to what is observed with Six1 overexpression. P values represent statistical analysis using a paired t test (*≦0.05, **≦0.01, ***≦0.001).

FIG. 6A-E. The miR-106b-25 cluster increases TIC characteristics. (FIG. 6A) Overexpression of the miR-106b-25 cluster in MCF7 cells is sufficient to increase the CD24low/CD44+ population, similar to what is observed with Six1 overexpression in MCF7 cells. (FIG. 6B) Expression of the miR-106b-25 cluster in MCF7 cells is sufficient to increase tumorsphere formation, a measurement of self-renewal capability, similar to what is observed with Six1 overexpression in MCF7 cells. (FIG. 6C) MCF7-Cluster cells transplanted into the 4th mammary fat pad of NOD-SCID mice at limiting dilutions have an increased ability to initiate tumors when compared to MCF7-NS cells. (FIG. 6D) Inhibition of the miR-106b-25 cluster in MCF7-Six1 cells (MCF7-Six1-Zip-Cluster) reduces the ability of the cells to initiate tumors, back to levels observed in MCF7-Ctrl cells (MCF7-Ctrl-Zip-SCR). (FIG. 6E) Genes important for stem cell maintenance, growth, and differentiation are increased in MCF7-Cluster cells as compared to MCF7-NS cells, as determined by a stem cell qRT-PCR array. Data is represented as-fold change in gene expression in MCF7-Cluster cells as compared to control MCF7-NS cells from 3 replicate plates of each condition.

FIGS. 7A-D. The miR-106b-25 cluster correlates with Six1 expression and activated TGF-β signaling in human breast cancers. Human breast cancer tissue arrays were previously immunostained with an anti-Six1 antibody (Atlas) and a Smad3 antibody (Zymed) as previously described (2) and the staining was scored for nuclear Six1 and Smad3 on a scale of 0-4. A serial section array was also stained for miR-106b expression by in situ hybridization. Expression of miR-106b was scored on a scale of 0-4 and compared to Six1 and nuclear Smad3 scores in the same tissues. (FIG. 7A) Results show that miR-106b and Six1 expression correlate in human breast cancers (FIG. 7B) as do miR-106b and nuclear Smad3. (FIG. 7C) When the expression of all three molecules is considered, the highest percentage of nuclear Smad3 can be found when both Six1 and miR-106b are highly expressed. P-values obtained using Spearman correlation analysis. (FIG. 7D) In a miRNA expression dataset of 216 early invasive breast cancers, 27 patients whose tumors express both high miR-106b and high miR-93 show a significantly reduced time to relapse. The median value for miR-106b and miR-93 was used to divide the samples into high (above median) and low (below median) miRNA expression. P value was calculated by log-rank analysis. A full color version of this figure is available at the Oncogene journal online.

### DESCRIPTION OF ILLUSTRATIVE EMBODIMENTS

Anti-TGF-β therapy aims to target both the tumor cell and the tumor microenvironment and may well have systemic effects of relevance to tumorigenesis. Extra-tumoral targets include stromal fibroblasts, endothelial and pericyte cells during angiogenesis, and the local and systemic immune systems, all of which can contribute to the pro-oncogenic effects of TGF-β. Many different approaches have been considered, such as interference with ligand synthesis using oligonucleotides, sequestration of extracellular ligand using naturally-occurring TGF-β binding proteins, recombinant proteins or antibodies, targeting activation of latent TGF-β at the cell surface, or signal transduction within the cell. Regardless, being able to apply these therapies only to the correct patient set would not only save considerable cost and wasted time, but it could also avoid the potential negative impact from side effects or even cancer progression. As reported below, the present inventors have now determined that several miRNAs linked to expression of the transcription factor Six1 apparently contribute to the tumor-promoting function of TGF-β. More directly, they can be used to identify patients who have undergone the switch from TGF-β tumor inhibition to TGF-β promotion. These and other aspects of the invention are set forth in detail below.

### I. TGF-BETA, SIX1 AND CANCER

In normal cells, TGF-β, acting through its signaling pathway, stops the cell cycle at the G1 stage to stop proliferation, induce differentiation, or promote apoptosis. When a cell is transformed into a cancer cell, parts of the TGF-β signaling pathway are mutated, and TGF-β no longer controls the cell. These cancer cells proliferate. The surrounding stromal cells (fibroblasts) also proliferate. Both types of cells increase their production of TGF-β. This TGF-β acts on the surrounding stromal cells, immune cells, endothelial and smooth-muscle cells. It causes immunosuppression and angiogenesis, which makes the cancer more invasive. TGF-β also converts effector T-cells, which normally attack cancer with an inflammatory (immune) reaction, into regulatory (suppressor) T-cells, which turn off the inflammatory reaction.

TGF-β does not work alone, however. The inventors have previous published on the effects of the regulatory factor Six1 on cancer development. Six1 belongs to the Six family of homeobox genes (Six1-6) (SEQ ID NOS: 4 and 5) encoding transcription factors that play vital roles in the development of many organs (Kawakami et al., 2000). Six1-6 share a DNA binding homeodomain (HD) and a Six domain (SD) responsible for co-activator binding (Kawakami et al., 2000). In particular, Six1 plays a role in cell growth, cell survival and cell migration during normal cell development. Six1 plays a critical role in the onset and progression of a significant proportion of breast and other cancers, but has never before been clinically targeted. The Six1 homeobox gene encodes a transcription factor that is crucial for the development of many organs but is down-regulated after organ development is complete. Its expression is low or undetectable in normal adult breast tissue but the gene is over-expressed in 50% of primary breast tumors and 90% of metastatic lesions. Examination of public microarray databases containing more than 535 breast cancer samples demonstrates that Six1 levels correlate significantly with shortened time to relapse, shortened time to metastasis, and decreased overall survival. In addition, Six1 overexpression correlates with adverse outcomes in numerous other cancers, including ovarian, hepatocellular carcinoma, and rhabdomyosarcoma. Using mouse models of mammary cancer, it was recently demonstrated that over-expression of Six1 results in enhanced proliferation, transformation, increased tumor volume, and metastasis. Importantly, RNA interference against Six1 decreases cancer cell proliferation and metastases in several different cancer models.

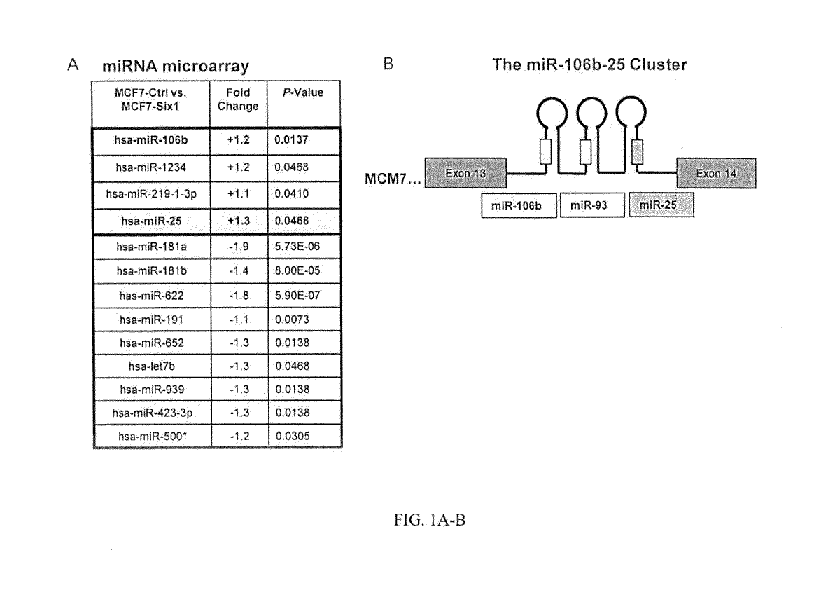

Six1 was shown to bind tightly to the MEF3 motif (TCAGGTT) (Spitz et al., 1998). This sequence is different from the TAAT core sequence bound by the canonical HD, likely due to the fact that the HD in Six1 differs from the “classic” HD at two highly conserved residues contacting DNA. The Six type HD is believed to confer a unique DNA binding specificity to the Six family members that differs from the TAAT core in the classic HD. However, the consensus Six1 recognition sequence remains unknown. A limited number of potential Six1 targets are identified (Kawakami et al., 1996; Spitz et al., 1998; Ando et al., 2005) and, indeed, none of them contain the TAAT core. Interestingly, these targets do not share an obvious consensus sequence, possibly due to the limited number of sequences analyzed. Recently, an ideal Six1 DNA binding sequence (TGATAC) was identified using combined bioinformatic and biologic approaches (Noyes et al., 2008; Berger et al., 2008). The Six1 target most relevant to breast tumorigenesis is the cyclin A1 promoter (Coletta et al., 2004). The transcriptional up-regulation of cyclin A1 by Six1 leads to an increase in proliferation in mammary carcinoma cells and Six1 mediated cell cycle progression is dependent on cyclin A1 (Coletta et al., 2004). In addition to the HD, the Six family members contain a conserved and novel Six-domain (SD) (FIG. 1) (Oliver et al., 1995). The SD contributes to DNA binding as well as to protein interaction with cofactors (Kawakami et al., 2000; Oliver et al., 1995).

Six1 does not have an intrinsic activation or repression domain and requires the Eya coactivator proteins to activate transcription. The Eya proteins utilize their intrinsic phosphatase activity to switch the Six1 transcriptional complex from a repressor to an activator complex. The Six1-Eya interaction is essential for proliferation during embryonic development, and both Six1 and Eya2 have been independently implicated in the same types of cancer. Because the Eya co-activator contains a unique protein phosphatase domain whose activity is required to activate Six1, it may serve as a novel anti-cancer drug target. Eya knockout mice phenocopy Six1 knockout mice (Xu et al., 1999). Six1's activity on cellular proliferation was also found to be dependent on Eya (Li et al., 2003). As Six1 contributes to breast tumorigenesis by stimulating cellular proliferation, the interaction between Eya and Six1 may be critical for Six1-mediated tumorigenesis.

The inventors also previously identified a number of miRNAs that appear to be regulated by Six1, or at least connected to the expression of Six1 such that their levels fluctuate along with levels of Six1. miR-106b, miR-93 and miR-25, the so-called miR-106b-25 cluster, were all observed to be increased where Six1 is increased, and both miR-375 and miR-622 are decreased with an increase in Six1 expression. In work described herein, the inventors have now determined that Six1 upregulates the miR-106b-25 cluster of miRs, and these miRs in turn upmodulate TGF-β signaling. These events contribute to the TGF-β switch from tumor inhibiting to tumor promoting.

### II. MIRNAS

A. Background

In 2001, several groups used a novel cloning method to isolate and identify a large group of “microRNAs” (miRNAs) from C. elegans, Drosophila, and humans (Lagos-Quintana et al., 2001; Lau et al., 2001; Lee and Ambros, 2001). Several hundreds of miRNAs have been identified in plants and animals—including humans—which do not appear to have endogenous siRNAs. Thus, while similar to siRNAs, miRNAs are nonetheless distinct.

miRNAs thus far observed have been approximately 21-22 nucleotides in length and they arise from longer precursors, which are transcribed from non-protein-encoding genes. See review of Carrington et al. (2003). The precursors form structures that fold back on each other in self-complementary regions; they are then processed by the nuclease Dicer in animals or DCL1 in plants. miRNA molecules interrupt translation through precise or imprecise base-pairing with their targets.

miRNAs are transcribed by RNA polymerase II and can be derived from individual miRNA genes, from introns of protein coding genes, or from poly-cistronic transcripts that often encode multiple, closely related miRNAs. Pre-miRNAs, generally several thousand bases long are processed in the nucleus by the RNase Drosha into 70- to 100-nt hairpin-shaped precursors. Following transport to the cytoplasm, the hairpin is further processed by Dicer to produce a double-stranded miRNA. The mature miRNA strand is then incorporated into the RNA-induced silencing complex (RISC), where it associates with its target mRNAs by base-pair complementarity. In the relatively rare cases in which a miRNA base pairs perfectly with an mRNA target, it promotes mRNA degradation. More commonly, miRNAs form imperfect heteroduplexes with target mRNAs, affecting either mRNA stability or inhibiting mRNA translation.

The 5′ portion of a miRNA spanning bases 2-8, termed the ‘seed’ region, is especially important for target recognition (Krenz and Robbins, 2004; Kiriazis and Krania, 2000). The sequence of the seed, together with phylogenetic conservation of the target sequence, forms the basis for many current target prediction models. Although increasingly sophisticated computational approaches to predict miRNAs and their targets are becoming available, target prediction remains a major challenge and requires experimental validation. Ascribing the functions of miRNAs to the regulation of specific mRNA targets is further complicated by the ability of individual miRNAs to base pair with hundreds of potential high and low affinity mRNA targets and by the targeting of multiple miRNAs to individual mRNAs.

The first miRNAs were identified as regulators of developmental timing in C. elegans, suggesting that miRNAs, in general, might play decisive regulatory roles in transitions between different developmental states by switching off specific targets (Fatkin et al., 2000; Lowes et al., 1997). However, subsequent studies suggest that miRNAs, rather than functioning as on-off “switches,” more commonly function to modulate or fine-tune cell phenotypes by repressing expression of proteins that are inappropriate for a particular cell type, or by adjusting protein dosage. miRNAs have also been proposed to provide robustness to cellular phenotypes by eliminating extreme fluctuations in gene expression (Miyata et al., 2000).

Research on microRNAs is increasing as scientists are beginning to appreciate the broad role that these molecules play in the regulation of eukaryotic gene expression. The two best understood miRNAs, lin-4 and let-7, regulate developmental timing in C. elegans by regulating the translation of a family of key mRNAs (reviewed in Pasquinelli, 2002). Several hundred miRNAs have been identified in C. elegans, Drosophila, mouse, and humans. As would be expected for molecules that regulate gene expression, miRNA levels have been shown to vary between tissues and developmental states. In addition, one study shows a strong correlation between reduced expression of two miRNAs and chronic lymphocytic leukemia, providing a possible link between miRNAs and cancer (Calin et al., 2002). Although the field is still young, there is speculation that miRNAs could be as important as transcription factors in regulating gene expression in higher eukaryotes.

There are a few examples of miRNAs that play critical roles in cell differentiation, early development, and cellular processes like apoptosis and fat metabolism. lin-4 and let-7 both regulate passage from one larval state to another during C. elegans development (Ambros, 2003). mir-14 and bantam are drosophila miRNAs that regulate cell death, apparently by regulating the expression of genes involved in apoptosis (Brennecke et al., 2003, Xu et al., 2003). MiR14 has also been implicated in fat metabolism (Xu et al., 2003). Lsy-6 and miR-273 are C. elegans miRNAs that regulate asymmetry in chemosensory neurons (Chang et al., 2004). Another animal miRNA that regulates cell differentiation is miR-181, which guides hematopoietic cell differentiation (Chen et al., 2004). These molecules represent the full range of animal miRNAs with known functions. Enhanced understanding of the functions of miRNAs will undoubtedly reveal regulatory networks that contribute to normal development, differentiation, inter- and intra-cellular communication, cell cycle, angiogenesis, apoptosis, and many other cellular processes. Given their important roles in many biological functions, it is likely that miRNAs will offer important points for therapeutic intervention or diagnostic analysis.

Characterizing the functions of biomolecules like miRNAs often involves introducing the molecules into cells or removing the molecules from cells and measuring the result. If introducing a miRNA into cells results in apoptosis, then the miRNA undoubtedly participates in an apoptotic pathway. Methods for introducing and removing miRNAs from cells have been described. Two recent publications describe antisense molecules that can be used to inhibit the activity of specific miRNAs (Meister et al., 2004; Hutvagner et al., 2004). Another publication describes the use of plasmids that are transcribed by endogenous RNA polymerases and yield specific miRNAs when transfected into cells (Zeng et al., 2002). These two reagent sets have been used to evaluate single miRNAs.

B. miR-106b
(SEQ ID NO: 1
The sequence for miR-106b is
UAAAGUGCUGACAGUGCAGAU.
C. miR-93
(SEQ ID NO: 2)
The sequence for miR-93 is
CAAAGUGCUGUUCGUGCAGGUAG.
D. miR-25
(SEQ ID NO: 3)
The sequence for miR-25 is
CAUUGCACUUGUCUCGGUCUGA.

E. Antagomirs

In certain embodiments, it may be desirable to also employ inhibitors of the foregoing miRNA in order to restore TGF-β's tumor inhibiting function. In general, such inhibitors of miRNAs take the form of “antagomirs,” short, chemically-engineered single-stranded oligonucleotides complementary to miRNAs that block their function (Krützfeldt et al., 2005). Other approaches include inhibition of miRNAs with antisense 2′-O-methyl (2′-OMe) oligoribonucleotides and small interfering double-stranded RNAs (siRNAs) engineered with certain “drug-like” properties (chemical modifications for stability; cholesterol conjugation for delivery) (Krützfeldt et al., 2005). Such therapies would not typically be combined with an anti-TGF-β.

F. Synthesis and Alternative Nucleic Acid Chemistries

Oligonucleotides are chemically synthesized using nucleoside phosphoramidites. A phosphoramidite is a derivative of natural or synthetic nucleoside with protection groups added to its reactive exocyclic amine and hydroxy groups. The naturally occurring nucleotides (nucleoside-3′-phosphates) are insufficiently reactive to afford the synthetic preparation of oligonucleotides. A dramatically more reactive (2-cyanoethyl) N,N-diisopropyl phosphoramidite group is therefore attached to the 3′-hydroxy group of a nucleoside to form nucleoside phosphoramidite. The protection groups prevent unwanted side reactions or facilitate the formation of the desired product during synthesis. The 5′-hydroxyl group is protected by DMT (dimethoxytrityl) group, the phosphite group by a diisopropylamino (iPr2N) group and a 2-cyanoethyl (OCH₂CH₂CN) group. The nucleic bases also have protecting groups on the exocyclic amine groups (benzoyl, acetyl, isobutyryl, or many other groups). In RNA synthesis, the 2′ group is protected with a TBDMS (t-butyldimethylsilyl) group or with a TOM (t-butyldimethylsilyloxymethyl) group. With the completion of the synthesis process, all the protection groups are removed.

Whereas enzymes synthesize DNA in a 5′ to 3′ direction, chemical DNA synthesis is done backwards in a 3′ to 5′ reaction. Based on the desired nucleotide sequence of the product, the phosphoramidites of nucleosides A, C, G, and T are added sequentially to react with the growing chain in a repeating cycle until the sequence is complete. In each cycle, the product's 5′-hydroxy group is deprotected and a new base is added for extension. In solid-phase synthesis, the oligonucleotide being assembled is bound, via its 3′-terminal hydroxy group, to a solid support material on which all reactions take place. The 3′ group of the first base is immobilized via a linker onto a solid support (most often, controlled pore glass particles or macroporouspolystyrene beads). This allows for easy addition and removal of reactants. In each cycle, several solutions containing reagents required for the elongation of the oligonucleotide chain by one nucleotide residue are sequentially pumped through the column from an attached reagent delivery system and removed by washing with an inert solvent.

Antagomirs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism. Modifications can also increase sequence specificity, and consequently decrease off-site targeting. In one embodiment, the antagomir includes a non-nucleotide moiety, e.g., a cholesterol moiety. The non-nucleotide moiety can be attached to the 3′ or 5′ end of the oligonucleotide agent.

A wide variety of well-known, alternative oligonucleotide chemistries may be used (see, e.g., U.S. Patent Publications 2007/0213292, 2008/0032945, 2007/0287831, etc.), particularly single-stranded complementary oligonucleotides comprising 2′ methoxyethyl, 2′-fluoro, and morpholino bases (see e.g., Summerton and Weller, 1997). The oligonucleotide may include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA). Also contemplated are locked nucleic acid (LNA) and peptide nucleic acids (PNA).

Peptide nucleic acids (PNAs) are nonionic DNA mimics that have outstanding potential for recognizing duplex DNA (Kaihatsu et al., 2004; Nielsen et al., 1991). PNAs can be readily synthesized and bind to complementary sequences by standard Watson-Crick base-pairing (Egholm et al., 1993), allowing them to target any sequence within the genome without the need for complex synthetic protocols or design considerations. Strand invasion of duplex DNA by PNAs is not hindered by phosphate-phosphate repulsion and is both rapid and stable (Kaihatsu et al., 2004; Nielsen et al., 1991). Applications for strand invasion by PNAs include creation of artificial primosomes (Demidov et al., 2001), inhibition of transcription (Larsen and Nielsen, 1996), activation of transcription (Mollegaard et al., 1994), and directed mutagenesis (Faruqi et al., 1998). PNAs would provide a general and potent strategy for probing the structure and function of chromosomal DNA in living systems if their remarkable strand invasion abilities could be efficiently applied inside cells.

Strand invasion by PNAs in cell-free systems is most potent at sequences that are partially single-stranded (Bentin and Nielsen, 1996; Zhang et al., 2000). Assembly of RNA polymerase and transcription factors into the pre-initiation complex on DNA induces the formation of a structure known as the open complex that contains several bases of single-stranded DNA (Holstege et al., 1997; Kahl et al., 2000). The exceptional ability of PNAs to recognize duplex DNA allows them to intercept the open complex of an actively transcribed gene without a requirement for preincubation. The open complex is formed during transcription of all genes and PNAs can be synthesized to target any transcription initiation site. Therefore, antigene PNAs that target an open complex at a promoter region within chromosomal DNA would have the potential to be general tools for controlling transcription initiation inside cells.

A locked nucleic acid (LNA), often referred to as inaccessible RNA, is a modified RNA nucleotide (Elmén et al., 2008). The ribose moiety of an LNA nucleotide is modified with an extra bridge connecting the 2′ and 4′ carbons. The bridge “locks” the ribose in the 3′-endo structural conformation, which is often found in the A-form of DNA or RNA. LNA nucleotides can be mixed with DNA or RNA bases in the oligonucleotide whenever desired. Such oligomers are commercially available. The locked ribose conformation enhances base stacking and backbone pre-organization. This significantly increases the thermal stability (melting temperature) of oligonucleotides (Kaur et al., 2006). LNA bases may be included in a DNA backbone, by they can also be in a backbone of LNA, 2′-O-methyl RNA, 2′-methoxyethyl RNA, or 2′-fluoro RNA. These molecules may utilize either a phosphodiester or phosphorothioate backbone.

Other oligonucleotide modifications can be made to produce oligonucleotides. For example, stability against nuclease degradation has been achieved by introducing a phosphorothioate (P═S) backbone linkage at the 3′ end for exonuclease resistance and 2′ modifications (2′-OMe, 2′-F and related) for endonuclease resistance (WO 2005115481; Li et al., 2005; Choung et al., 2006). A motif having entirely of 2′-O-methyl and 2′-fluoro nucleotides has shown enhanced plasma stability and increased in vitro potency (Allerson et al., 2005). The incorporation of 2′-O-Me and 2′-O-MOE does not have a notable effect on activity (Prakash et al., 2005).

Sequences containing a 4′-thioribose modification have been shown to have a stability 600 times greater than that of natural RNA (Hoshika et al, 2004). Crystal structure studies reveal that 4′-thioriboses adopt conformations very similar to the C3′-endo pucker observed for unmodified sugars in the native duplex (Haeberli et al., 2005). Stretches of 4′-thio-RNA were well tolerated in both the guide and nonguide strands. However, optimization of both the number and the placement of 4′-thioribonucleosides is necessary for maximal potency.

In the boranophosphate linkage, a non-bridging phosphodiester oxygen is replaced by an isoelectronic borane (BH3-) moiety. Boranophosphate siRNAs have been synthesized by enzymatic routes using T7 RNA polymerase and a boranophosphate ribonucleoside triphosphate in the transcription reaction. Boranophosphate siRNAs are more active than native siRNAs if the center of the guide strand is not modified, and they may be at least ten times more nuclease resistant than unmodified siRNAs (Hall et al., 2004; Hall et al., 2006).

Certain terminal conjugates have been reported to improve or direct cellular uptake. For example, NAAs conjugated with cholesterol improve in vitro and in vivo cell permeation in liver cells (Rand et al., 2005). Soutschek et al. (2004) have reported on the use of chemically-stabilized and cholesterol-conjugated siRNAs have markedly improved pharmacological properties in vitro and in vivo. Chemically-stabilized siRNAs with partial phosphorothioate backbone and 2′-O-methyl sugar modifications on the sense and antisense strands (discussed above) showed significantly enhanced resistance towards degradation by exo- and endonucleases in serum and in tissue homogenates, and the conjugation of cholesterol to the 3′ end of the sense strand of an oligonucleotides by means of a pyrrolidine linker does not result in a significant loss of gene-silencing activity in cell culture. These study demonstrates that cholesterol conjugation significantly improves in vivo pharmacological properties of oligonucleotides.

U.S. Patent Publication 2008/0015162, incorporated herein by reference, provide additional examples of nucleic acid analogs useful in the present invention. The following excerpts are derived from that document and are exemplary in nature only.

In certain embodiments, oligomeric compounds comprise one or more modified monomers, including 2′-modified sugars, such as BNA's and monomers (e.g., nucleosides and nucleotides) with 2′-substituents such as allyl, amino, azido, thio, O-allyl, O—C₁-C₁₀ alkyl, —OCF₃, O—(CH₂)₂—O—CH₃, 2′—O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl.

In certain embodiments, the oligomeric compounds including, but no limited to short oligomers of the present invention, comprise one or more high affinity monomers provided that the oligomeric compound does not comprise a nucleotide comprising a 2′—O(CH₂)ₙH, wherein n is one to six. In certain embodiments, the oligomeric compounds including, but no limited to short oligomers of the present invention, comprise one or more high affinity monomer provided that the oligomeric compound does not comprise a nucleotide comprising a 2′-OCH₃ or a 2′-O(CH₂)₂OCH₃. In certain embodiments, the oligomeric compounds comprise one or more high affinity monomers provided that the oligomeric compound does not comprise a α-L-methyleneoxy (4′-CH₂—O-2′) BNA and/or a β-D-methyleneoxy (4′-CH₂—O-2′) BNA.

Certain BNAs have been prepared and disclosed in the patent literature as well as in scientific literature (Singh et al., 1998; Koshkin et al., 1998; Wahlestedt et al., 2000; Kumar et al., 1998; WO 94/14226; WO 2005/021570; Singh et al, 1998; examples of issued US patents and published applications that disclose BNAs include, for example, U.S. Pat. Nos. 7,053,207; 6,268,490; 6,770,748; 6,794,499; 7,034,133; and 6,525,191; and U.S. Patent Publication Nos. 2004/0171570; 2004/0219565; 2004/0014959; 2003/0207841; 2004/0143114; and 2003/0082807.

Also provided herein are BNAs in which the 2′-hydroxyl group of the ribosyl sugar ring is linked to the 4′ carbon atom of the sugar ring thereby forming a methyleneoxy (4′-CH₂—O-2′) linkage to form the bicyclic sugar moiety (reviewed in Elayadi et al., 2001; Braasch et al., 2001; see also U.S. Pat. Nos. 6,268,490 and 6,670,461). The linkage can be a methylene (—CH₂—) group bridging the 2′ oxygen atom and the 4′ carbon atom, for which the term methyleneoxy (4′-CH₂—O-2′) BNA is used for the bicyclic moiety; in the case of an ethylene group in this position, the term ethyleneoxy (4′-CH₂CH₂—O-2′) BNA is used (Singh et al., 1998; Morita et al., 2003). Methyleneoxy (4′-CH₂—O-2′) BNA and other bicyclic sugar analogs display very high duplex thermal stabilities with complementary DNA and RNA (Tm=+3 to +10° C.), stability towards 3′-exonucleolytic degradation and good solubility properties. Potent and nontoxic antisense oligonucleotides comprising BNAs have been described (Wahlestedt et al., 2000).

An isomer of methyleneoxy (4′-CH₂—O-2′) BNA that has also been discussed is α-L-methyleneoxy (4′-CH₂—O-2′) BNA which has been shown to have superior stability against a 3′-exonuclease. The α-L-methyleneoxy (4′-CH₂—O-2′) BNA's were incorporated into antisense gapmers and chimeras that showed potent antisense activity (Frieden et al., 2003).

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., 1998). BNAs and preparation thereof are also described in WO 98/39352 and WO 99/14226.

Analogs of methyleneoxy (4′-CH₂—O-2′) BNA, phosphorothioate-methyleneoxy (4′-CH₂—O-2′) BNA and 2′-thio-BNAs, have also been prepared (Kumar et al., 1998). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., 1998). In addition, 2′-amino- and 2′-methylamino-BNA's have been prepared and the thermal stability of their duplexes with complementary RNA and DNA strands has been previously reported.

Modified sugar moieties are well known and can be used to alter, typically increase, the affinity of oligomers for targets and/or increase nuclease resistance. A representative list of modified sugars includes, but is not limited to, bicyclic modified sugars (BNA's), including methyleneoxy (4′-CH₂—O-2′) BNA and ethyleneoxy (4′-(CH₂)₂—O-2′ bridge) BNA; substituted sugars, especially 2′-substituted sugars having a 2′-F, 2′-OCH₃ or a 2′-O(CH₂)₂—OCH₃ substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

The naturally-occurring base portion of a nucleoside is typically a heterocyclic base. The two most common classes of such heterocyclic bases are the purines and the pyrimidines. For those nucleosides that include a pentofuranosyl sugar, a phosphate group can be linked to the 2′, 3′ or 5′ hydroxyl moiety of the sugar. In forming oligonucleotides, those phosphate groups covalently link adjacent nucleosides to one another to form a linear polymeric compound. Within oligonucleotides, the phosphate groups are commonly referred to as forming the internucleotide backbone of the oligonucleotide. The naturally occurring linkage or backbone of RNA and of DNA is a 3′ to 5′ phosphodiester linkage.

In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable with the compounds described herein. In certain embodiments, a modified nucleobase is a nucleobase that is fairly similar in structure to the parent nucleobase, such as for example a 7-deaza purine, a 5-methyl cytosine, or a G-clamp. In certain embodiments, nucleobase mimetic include more complicated structures, such as for example a tricyclic phenoxazine nucleobase mimetic. Methods for preparation of the above noted modified nucleobases are well known to those skilled in the art.

Described herein are linking groups that link monomers (including, but not limited to, modified and unmodified nucleosides and nucleotides) together, thereby forming an oligomeric compound. The two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂—), thiodiester (—O—C(O)—S—), thionocarbamate (—O—C(O)(NH)—S—); siloxane (—O—Si(H)₂—O—); and N,N′-dimethylhydrazine (—CH₂—N(CH₃)—N(CH₃)—). Oligomeric compounds having non-phosphorus linking groups are referred to as oligonucleosides. Modified linkages, compared to natural phosphodiester linkages, can be used to alter, typically increase, nuclease resistance of the oligomeric compound. In certain embodiments, linkages having a chiral atom can be prepared a racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

G. Delivery

A variety of methods may be used to deliver oligonucleotides, including antagomirs and mimics, into a target cell. For cells in vitro embodiments, delivery can often be accomplished by direct injection into cells, and delivery can often be enhanced using hydrophobic or cationic carriers. Alternatively, the cells can be permeabilized with a permeabilization and then contacted with the oligonucleotide. The antagomir can be administered to the subject either as a naked oligonucleotide agent, in conjunction with a delivery reagent, or as a recombinant plasmid or viral vector which expresses the oligonucleotide agent.

For cells in situ, several applicable delivery methods are well-established, e.g., Elmén et al. (2008), Akinc et al. (2008); Esau et al. (2006), Krützfeldt et al. (2005). In particular, cationic lipids (see e.g., Hassani et al., 2004) and polymers such as polyethylenimine (see e.g., Urban-Klein, 2005) have been used to facilitate oligonucleotide delivery. Compositions consisting essentially of the oligomer (i.e., the oligomer in a carrier solution without any other active ingredients) can be directly injected into the host (see e.g., Tyler et al., 1999; McMahon et al., 2002). In vivo applications of duplex RNAs are reviewed in Paroo and Corey (2004).

When microinjection is not an option, delivery can be enhanced in some cases by using Lipofectamine™ (Invitrogen, Carlsbad, Calif.). PNA oligomers can be introduced into cells in vitro by complexing them with partially complementary DNA oligonucleotides and cationic lipid. The lipid promotes internalization of the DNA, while the PNA enters as cargo and is subsequently released. Peptides such as penetratin, transportan, Tat peptide, nuclear localization signal (NLS), and others, can be attached to the oligomer to promote cellular uptake (see e.g., Kaihatsu et al., 2003; Kaihatsu et al., 2004). Alternatively, the cells can be permeabilized with a permeabilization agent such as lysolecithin, and then contacted with the oligomer.

Alternatively, certain single-stranded oligonucleotide agents featured in the instant invention can be expressed within cells from eukaryotic promoters (e.g., Izant and Weintraub, 1985; McGarry and Lindquist, 1986; Scanlon et al., 1991; Kashani-Sabet et al., 1992; Weerasinghe et al., 1991; Ojwang et al., 1992; Chen et al., 1992; Sarver et al., 1990; Thompson et al., 1995). Those skilled in the art realize that any nucleic acid can be expressed in eukaryotic cells from the appropriate DNA/RNA vector. The activity of such nucleic acids can be augmented by their release from the primary transcript by a enzymatic nucleic acid (PCT WO 93/23569; PCT WO 94/02595; Ohkawa et al., 1992; Taira et al., 1991; Ventura et al., 1993; Chowrira et al., 1994).

The recombinant vectors can be DNA plasmids or viral vectors. Oligonucleotide agent-expressing viral vectors can be constructed based on, but not limited to, adeno-associated virus, retrovirus, adenovirus, or alphavirus. In another embodiment, pol III based constructs are used to express nucleic acid molecules of the invention (see for example Morris et al., 2004; U.S. Pat. Nos. 5,902,880 and 6,146,886). The recombinant vectors capable of expressing the oligonucleotide agents can be delivered as described above, and can persist in target cells. Alternatively, viral vectors can be used that provide for transient expression of nucleic acid molecules. Such vectors can be repeatedly administered as necessary. Once expressed, the antagomir interacts with the target RNA (e.g., miRNA or pre-miRNA) and inhibits miRNA activity. In a particular embodiment, the antagomir forms a duplex with the target miRNA, which prevents the miRNA from binding to its target mRNA, which results in increased translation of the target mRNA. Delivery of oligonucleotide agent-expressing vectors can be systemic, such as by intravenous or intra-muscular administration, by administration to target cells ex-planted from a subject followed by reintroduction into the subject, or by any other means that would allow for introduction into the desired target cell (see Couture et al., 1996).

Methods for the delivery of nucleic acid molecules are also described in Akhtar et al. (1992), Akhtar (1995), Maurer et al. (1999), Hofland and Huang (1999), Lee et al. (2000), all of which are incorporated herein by reference. U.S. Pat. No. 6,395,713 and PCT WO 94/02595 and WO 00/53722 further describe general methods for delivery of nucleic acid molecules.

### III. DETECTION OF NUCLEIC ACIDS

Nucleic acids can used be as probes or primers for embodiments involving nucleic acid hybridization. As such, they may be used to assess miR expression for the miR106b-25 cluster. Various aspects of nucleic acid detection as discussed below.

A. Hybridization

The use of a probe or primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

Accordingly, the nucleotide sequences of the invention may be used for their ability to selectively form duplex molecules with complementary stretches of DNAs and/or RNAs or to provide primers for amplification of DNA or RNA from samples. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications it is appreciated that lower stringency conditions are preferred. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In particular embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772 and U.S. Patent Publication 2008/0009439. The relevant portions of these and other references identified in this section of the Specification are incorporated herein by reference.

B. In Situ Hybridization

In situ hybridization (ISH) is a type of hybridization that uses a labeled complementary DNA or RNA strand (i.e., probe) to localize a specific DNA or RNA sequence in a portion or section of tissue (in situ), or, if the tissue is small enough (e.g. plant seeds, Drosophila embryos), in the entire tissue (whole mount ISH). This is distinct from immunohistochemistry, which localizes proteins in tissue sections. Fluorescent DNA ISH (FISH) can, for example, be used in medical diagnostics to assess chromosomal integrity. RNA ISH (hybridization histochemistry) is used to measure and localize mRNAs and other transcripts within tissue sections or whole mounts.

For hybridization histochemistry, sample cells and tissues are usually treated to fix the target transcripts in place and to increase access of the probe. As noted above, the probe is either a labeled complementary DNA or, now most commonly, a complementary RNA (riboprobe). The probe hybridizes to the target sequence at elevated temperature, and then the excess probe is washed away (after prior hydrolysis using RNase in the case of unhybridized, excess RNA probe). Solution parameters such as temperature, salt and/or detergent concentration can be manipulated to remove any non-identical interactions (i.e., only exact sequence matches will remain bound). Then, the probe that was labeled with either radio-, fluorescent- or antigen-labeled bases (e.g., digoxigenin) is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

C. Amplification of Nucleic Acids

Nucleic acids used as a template for amplification may be isolated from cells, tissues or other samples according to standard methodologies (Sambrook et al., 2001). In certain embodiments, analysis is performed on whole cell or tissue homogenates or biological fluid samples without substantial purification of the template nucleic acid. The nucleic acid may be genomic DNA or fractionated or whole cell RNA. Where RNA is used, it may be desired to first convert the RNA to a complementary DNA.

The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to any sequence corresponding to a nucleic acid sequence are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids containing one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemiluminescence, radioactive scintigraphy of incorporated radiolabel or fluorescent label or even via a system using electrical and/or thermal impulse signals (Bellus, 1994).

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al. (1988), each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 2001). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Reverse transcription (RT) of RNA to cDNA followed by quantitative PCR (RT-PCR) can be used to determine the relative concentrations of specific mRNA species isolated from a cell, such as a Six1 or Eya-encoding transcript. By determining that the concentration of a specific mRNA species varies, it is shown that the gene encoding the specific mRNA species is differentially expressed. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR amplification is directly proportional to the starting concentration of the target before the reaction began. By determining the concentration of the amplified products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR products and the relative mRNA abundances is only true in the linear range of the PCR reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR products must be sampled when the PCR reactions are in the linear portion of their curves.

A second condition for an RT-PCR experiment is to determine the relative abundances of a particular mRNA species. Typically, relative concentrations of the amplifiable cDNAs are normalized to some independent standard. The goal of an RT-PCR experiment is to determine the abundance of a particular mRNA species relative to the average abundance of all mRNA species in the sample.

Most protocols for competitive PCR utilize internal PCR standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

RT-PCR can be performed as a relative quantitative RT-PCR with an internal standard in which the internal standard is an amplifiable cDNA fragment that is larger than the target cDNA fragment and in which the abundance of the mRNA encoding the internal standard is roughly 5-100-fold higher than the mRNA encoding the target. This assay measures relative abundance, not absolute abundance of the respective mRNA species.

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989).

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 2001). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Various nucleic acid detection methods known in the art are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

D. Chip Technologies and Nucleic Acid Arrays

The present invention may involve the use of arrays or data generated from an array. Data may be readily available. Moreover, an array may be prepared in order to generate data that may then be used in correlation studies.

An array generally refers to ordered macroarrays or microarrays of nucleic acid molecules (probes) that are fully or nearly complementary or identical to a plurality of mRNA molecules or cDNA molecules and that are positioned on a support material in a spatially separated organization. Macroarrays are typically sheets of nitrocellulose or nylon upon which probes have been spotted. Microarrays position the nucleic acid probes more densely such that up to 10,000 nucleic acid molecules can be fit into a region typically 1 to 4 square centimeters. Microarrays can be fabricated by spotting nucleic acid molecules, e.g., genes, oligonucleotides, etc., onto substrates or fabricating oligonucleotide sequences in situ on a substrate. Spotted or fabricated nucleic acid molecules can be applied in a high density matrix pattern of up to about 30 non-identical nucleic acid molecules per square centimeter or higher, e.g. up to about 100 or even 1000 per square centimeter. Microarrays typically use coated glass as the solid support, in contrast to the nitrocellulose-based material of filter arrays. By having an ordered array of complementing nucleic acid samples, the position of each sample can be tracked and linked to the original sample. A variety of different array devices in which a plurality of distinct nucleic acid probes are stably associated with the surface of a solid support are known to those of skill in the art. Useful substrates for arrays include nylon, glass and silicon Such arrays may vary in a number of different ways, including average probe length, sequence or types of probes, nature of bond between the probe and the array surface, e.g. covalent or non-covalent, and the like. The labeling and screening methods of the present invention and the arrays are not limited in its utility with respect to any parameter except that the probes detect expression levels; consequently, methods and compositions may be used with a variety of different types of genes.

Representative methods and apparatus for preparing a microarray have been described, for example, in U.S. Pat. Nos. 5,143,854; 5,202,231; 5,242,974; 5,288,644; 5,324,633; 5,384,261; 5,405,783; 5,412,087; 5,424,186; 5,429,807; 5,432,049; 5,436,327; 5,445,934; 5,468,613; 5,470,710; 5,472,672; 5,492,806; 5,503,980; 5,510,270; 5,525,464; 5,527,681; 5,529,756; 5,532,128; 5,545,531; 5,547,839; 5,554,501; 5,556,752; 5,561,071; 5,571,639; 5,580,726; 5,580,732; 5,593,839; 5,599,695; 5,599,672; 5,610,287; 5,624,711; 5,631,134; 5,639,603; 5,654,413; 5,658,734; 5,661,028; 5,665,547; 5,667,972; 5,695,940; 5,700,637; 5,744,305; 5,800,992; 5,807,522; 5,830,645; 5,837,196; 5,871,928; 5,847,219; 5,876,932; 5,919,626; 6,004,755; 6,087,102; 6,368,799; 6,383,749; 6,617,112; 6,638,717; 6,720,138, as well as WO 93/17126; WO 95/11995; WO 95/21265; WO 95/21944; WO 95/35505; WO 96/31622; WO 97/10365; WO 97/27317; WO 99/35505; WO 09923256; WO 09936760; WO0138580; WO 0168255; WO 03020898; WO 03040410; WO 03053586; WO 03087297; WO 03091426; WO03100012; WO 04020085; WO 04027093; EP 373 203; EP 785 280; EP 799 897 and UK 8 803 000; the disclosures of which are all herein incorporated by reference.

It is contemplated that the arrays can be high density arrays, such that they contain 100 or more different probes. It is contemplated that they may contain 1000, 16,000, 65,000, 250,000 or 1,000,000 or more different probes. The probes can be directed to targets in one or more different organisms. The oligonucleotide probes range from 5 to 50, 5 to 45, 10 to 40, or 15 to 40 nucleotides in length in some embodiments. In certain embodiments, the oligonucleotide probes are 20 to 25 nucleotides in length.

The location and sequence of each different probe sequence in the array are generally known. Moreover, the large number of different probes can occupy a relatively small area providing a high density array having a probe density of generally greater than about 60, 100, 600, 1000, 5,000, 10,000, 40,000, 100,000, or 400,000 different oligonucleotide probes per cm². The surface area of the array can be about or less than about 1, 1.6, 2, 3, 4, 5, 6, 7, 8, 9, or 10 cm².

Moreover, a person of ordinary skill in the art could readily analyze data generated using an array. Such protocols are disclosed above, and include information found in WO 9743450; WO 03023058; WO 03022421; WO 03029485; WO 03067217; WO 03066906; WO 03076928; WO 03093810; WO 03100448A1, all of which are specifically incorporated by reference.

### IV. METHODS OF THERAPY

In some embodiments, the invention provides compositions and methods for the treatment of cancer. In one embodiment, the invention provides a method of treating cancer comprising administering to a patient an anti-TGF-β therapy. This treatment may be further combined with additional cancer treatments. One of skill in the art will be aware of many treatments that may be combined with the methods of the present invention, some but not all of which are described below.

In general, the cancers will be characterized by overexpression of Six1, although they are not so limited. Thus, it is contemplated that a wide variety of tumors may be treated using anti-TGF-β therapies, including cancers of the brain, lung, liver, spleen, kidney, lymph node, pancreas, small intestine, blood cells, colon, stomach, breast, endometrium, prostate, testicle, ovary, skin, head and neck, esophagus, bone marrow, blood or other tissue.

In many contexts, it is not necessary that the tumor cell be killed or induced to undergo normal cell death or “apoptosis.” Rather, to accomplish a meaningful treatment, all that is required is that the tumor growth be slowed to some degree. It may be that the tumor growth is completely blocked, however, or that some tumor regression is achieved. Clinical terminology such as “remission” and “reduction of tumor” burden also are contemplated given their normal usage.

A. Antibodies

A number of anti-TGF-β antibodies are commercially available and are currently in use. One, designated GC1008, is currently being developed for treatment of kidney cancer, melanoma, and pulmonary fibrosis. However, one of skill in the art can prepare similar antibodies using standard methods of preparing and characterizing antibodies known in the art (see, e.g., Antibodies: A Laboratory Manual, Cold Spring Harbor Laboratory, 1988; U.S. Pat. No. 4,196,265). The methods for generating monoclonal antibodies (MAbs) generally begin along the same lines as those for preparing polyclonal antibodies. The first step for both these methods is immunization of an appropriate host or identification of subjects who are immune due to prior natural infection. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens or lymph nodes, or from circulating blood. The antibody-producing B lymphocytes from the immunized animal are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, pp. 65-66, 1986; Campbell, pp. 75-83, 1984). For example, where the immunized animal is a mouse, one may use P3-X63/Ag8, X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with human cell fusions. One particular murine myeloma cell is the NS-1 myeloma cell line (also termed P3-NS-1-Ag4-1), which is readily available from the NIGMS Human Genetic Mutant Cell Repository by requesting cell line repository number GM3573. Another mouse myeloma cell line that may be used is the 8-azaguanine-resistant mouse murine myeloma SP2/0 non-producer cell line. More recently, additional fusion partner lines for use with human B cells have been described, including KR12 (ATCC CRL-8658; K6H6/B5 (ATCC CRL-1823 SHM-D33 (ATCC CRL-1668) and HMMA2.5 (Posner et al., 1987). The antibodies in this invention were generated using the HMMA2.5 line.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986). The hybridomas secreting the influenza antibodies in this invention were obtained by electrofusion.

Fusion procedures usually produce viable hybrids at low frequencies, about 1×10⁻⁶ to 1×10⁻⁸. However, this does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine. Ouabain is added if the B cell source is an Epstein Barr virus (EBV) transformed human B cell line, in order to eliminate EBV transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain is also used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like.

The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the invention can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present invention can be synthesized using an automated peptide synthesizer.

It also is contemplated that a molecular cloning approach may be used to generate monoclonals. For this, RNA can be isolated from the hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using antigens. The advantages of this approach over conventional hybridoma techniques are that approximately 10⁴ times as many antibodies can be produced and screened in a single round, and that new specificities are generated by H and L chain combination which further increases the chance of finding appropriate antibodies. This also facilitates transfer of CDRs or entire variable regions into human framework/constant regions to create “humanized” antibodies where the original antibody is from a non-human source (i.e., mouse).

Other U.S. patents, each incorporated herein by reference, that teach the production of antibodies useful in the present invention include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antisense

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

C. Interfering RNAs

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis;

through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

A small hairpin RNA or short hairpin RNA (shRNA) is a sequence of RNA that makes a tight hairpin turn that can be used to silence gene expression via RNA interference. shRNA uses a vector introduced into cells and utilizes the U6 or H1 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it.

shRNA is transcribed by RNA polymerase III. shRNA production in a mammalian cell can sometimes cause the cell to mount an interferon response as the cell seeks to defend itself from what it perceives as viral attack. This problem is not observed in miRNA, which is transcribed by RNA polymerase II (the same polymerase used to transcribe mRNA).

shRNAs can also be made for use in plants and other systems, and are not necessarily driven by a U6 promoter. In plants the traditional promoter for strong constitutive expression (in most plant species) is the cauliflower mosaic virus 35S promoter (CaMV35S), in which case RNA Polymerase II is used to express the transcript destined to initiate RNAi.

D. Small Molecules

A variety of small molecules are known that inhibit TGF-β function, including those designated SM16 (Fridlender et al., 2009) and SB-431542 (Laping et al., 2002). Zu et al. (2011) and Kelly & Morris (2010) provide reviews of the topic.

E. Formulations and Routes for Administration to Patients

In some embodiments, the invention provides a method of treating cancer comprising providing to a patient an effective amount of a Six1 miRNA. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intradermal, subcutaneous, intramuscular, intraperitoneal or intravenous injection. Such compositions would normally be administered as pharmaceutically acceptable compositions. Of particular interest is direct intratumoral administration, perfusion of a tumor, or administration local or regional to a tumor, for example, in the local or regional vasculature or lymphatic system, or in a resected tumor bed (e.g., post-operative catheter). For practically any tumor, systemic delivery also is contemplated. This will prove especially important for attacking microscopic or metastatic cancer.

The active compounds may also be administered as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The actual dosage amount of a composition of the present invention administered to a patient or subject can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

“Treatment” and “treating” refer to administration or application of a therapeutic agent to a subject or performance of a procedure or modality on a subject for the purpose of obtaining a therapeutic benefit of a disease or health-related condition.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

A “disease” can be any pathological condition of a body part, an organ, or a system resulting from any cause, such as infection, genetic defect, and/or environmental stress.

“Prevention” and “preventing” are used according to their ordinary and plain meaning to mean “acting before” or such an act. In the context of a particular disease, those terms refer to administration or application of an agent, drug, or remedy to a subject or performance of a procedure or modality on a subject for the purpose of blocking the onset of a disease or health-related condition.

The subject can be a subject who is known or suspected of being free of a particular disease or health-related condition at the time the relevant preventive agent is administered. The subject, for example, can be a subject with no known disease or health-related condition (i.e., a healthy subject).

In additional embodiments of the invention, methods include identifying a patient in need of treatment. A patient may be identified, for example, based on taking a patient history or based on findings on clinical examination.

F. Cancer Combination Treatments

In some embodiments, the method further comprises treating a patient with cancer with a conventional cancer treatment. This approach can be applied to improve the efficacy of chemo- and radiotherapy in combination with anti-TGF-β treatments. In the context of the present invention, it is contemplated that the secondary treatment could be, but is not limited to, chemotherapeutic, radiation, a polypeptide inducer of apoptosis or other therapeutic intervention. It also is conceivable that more than one administration of the treatment will be desired.

1. Chemotherapy

A wide variety of chemotherapeutic agents may be used in accordance with the present invention. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein transferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present invention may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of your internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

Another immunotherapy could also be used as part of a combined therapy with gen silencing therapy discussed above. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-12, GM-CSF, gamma-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies, e.g., anti-ganglioside GM2, anti-HER-2, anti-p185 (Pietras et al., 1998; Hanibuchi et al., 1998; U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present invention may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Gene Therapy

In yet another embodiment, the secondary treatment is a gene therapy in which a therapeutic polynucleotide is administered before, after, or at the same time as a Six1 or Eya inhibitor is administered. Delivery of a Six1 or Eya inhibitor in conjunction with a vector encoding one of the following gene products may have a combined anti-hyperproliferative effect on target tissues. A variety of proteins are encompassed within the invention, some of which are described below.

a. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA or siRNA directed to a particular inducer of cellular proliferation is used to prevent expression of the inducer of cellular proliferation.

The proteins FMS and ErbA are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

b. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, mda-7, FHIT, p16 and C-CAM can be employed.

In addition to p53, another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/Six1 or Eya2, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

c. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

6. Other Agents

It is contemplated that other agents may be used with the present invention. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon α, β, and γ; IL-2 and other cytokines; F42K and other cytokine analogs; or MIP-1, MIP-1β, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

F. Dosage, Routes and Regimens

An anti-TGF-β agent can be administered at a unit dose less, depending on the type of agent. The unit dose, for example, can be administered by injection (e.g., intravenous or intramuscular, intrathecally, intratumorally or directly into an organ), inhalation, or a topical application. Significant modulation of TGF-β expression or activity may be achieved using nanomolar/submicromolar or picomolar/subnamomolar concentrations, and it is typical to use the lowest concentration possible to achieve the desired result.

In one embodiment, the unit dose is administered once a day, e.g., or less frequently less than or at about every 2, 4, 8 or 30 days. In another embodiment, the unit dose is not administered with a frequency (e.g., not a regular frequency). For example, the unit dose may be administered a single time. Because oligonucleotide agent can persist for several days after administering, in many instances, it is possible to administer the composition with a frequency of less than once per day, or, for some instances, only once for the entire therapeutic regimen. Where the administration by infusion, the infusion can be a single sustained dose or can be delivered by multiple infusions. Injection of agent can be directly into the tissue at or near the site of interest. Multiple injections of can be made into the tissue at or near the site.

In a particular dosage regimen, the agent is injected at or near a disease site once a day for seven days, for example, into a tumor, a tumor bed, or tumor vasculature. Where a dosage regimen comprises multiple administrations, it is understood that the effective amount of the agent administered to the subject can include the total amount of agent administered over the entire dosage regimen. One skilled in the art will appreciate that the exact individual dosages may be adjusted somewhat depending on a variety of factors, including the specific agent being administered, the time of administration, the route of administration, the nature of the formulation, the rate of excretion, the particular disorder being treated, the severity of the disorder, the pharmacodynamics of the oligonucleotide agent, and the age, sex, weight, and general health of the patient. Wide variations in the necessary dosage level are to be expected in view of the differing efficiencies of the various routes of administration. Variations in these dosage levels can be adjusted using standard empirical routines of optimization, which are well-known in the art. The precise therapeutically effective dosage levels and patterns can be determined by the attending physician in consideration of the above-identified factors.

In one embodiment, a subject is administered an initial dose, and one or more maintenance doses of an agent. The maintenance dose or doses are generally lower than the initial dose, e.g., one-half less of the initial dose. The maintenance doses are generally administered no more than once every 5, 10, or 30 days. Further, the treatment regimen may last for a period of time which will vary depending upon the nature of the particular disease, its severity and the overall condition of the patient. Following treatment, the patient can be monitored for changes in his condition and for alleviation of the symptoms of the disease state. The dosage of the compound may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered two or more doses, as desired or considered appropriate under the specific circumstances. If desired to facilitate repeated or frequent infusions, implantation of a delivery device, e.g., a pump, semi-permanent stent (e.g., intravenous, intraperitoneal, intracisternal or intracapsular), or reservoir may be advisable.

Certain factors may influence the dosage required to effectively treat a subject, including but not limited to the severity of the disease or disorder, previous treatments, the general health and/or age of the subject, and other diseases present. It will also be appreciated that the effective dosage of the agent used for treatment may increase or decrease over the course of a particular treatment. Changes in dosage may result and become apparent from the results of diagnostic assays. For example, the subject can be monitored after administering an agent. Based on information from the monitoring, an additional amount of the agent can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual compounds, and can generally be estimated based on EC₅₀'s found to be effective in in vitro and in vivo animal models.

### V. EXAMPLES

The following examples are included to demonstrate particular embodiments of the invention. It should be appreciated by those of skill in the art that the techniques disclosed in the examples which follow represent techniques discovered by the inventor to function well in the practice of the invention, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

microRNA Microarray and mRNA Microarray.

Total RNA was isolated using TRIzol reagent and analyzed using Agilent bioanalyzer. RNA preparations were sent to Thermo Scientific, where the miRNA microarray was performed. Twelve MCF7-Ctrl RNA samples and twelve MCF7-Six1 RNA samples were submitted, which represented 3 clonal isolates of each condition in replicates of four. miRNAs with a P-value <0.05 were employed for consideration of top miRNAs differential between MCF7-Ctrl and MCF7-Six1 cells. For mRNA microarray analysis, RNA was prepared in the same way as above, and submitted to the Genomics and Microarray Core at the University of Colorado Denver. The array was performed using the Affymetrix Human Exon 1.0 ST Array. Analysis of mRNA microarray results have been described previously (Micalizzi et al., 2009).

Cell Culture and Constructs.

Generation of MCF7-Ctrl and MCF7-Six1 cell lines was described previously (Ford and Pardee, 1998). MCF7-Cluster cells were generated by inserting the cloned miR-106b-25 cluster into the miR-Express vector (Open Biosystems) using XhoI and NotI restriction sites. Empty vector (EV) and non-silencing (NS) miR-express constructs were obtained from open biosystems. MCF7 cells were separately infected with lentivirus made from all miR-express constructs, and selected with 2.5 μg/mL puromycin. miRzip constructs were obtained from System Biosciences. The miRZip-cluster construct contains all three miRNA inhibitors (miRZip-106b, miRZip-93, miRZip-25) in the same vector. MCF7-Ctrl and MCF7-Six1 cells were infected with miRZip lentivirus, and selected with 2.5 μg/mL puromycin. All cell lines used in this study were fingerprinted to verify cell identity.

Real-Time PCR.

Total RNA was extracted with the miRNeasy RNA isolation kit (Qiagen) following the manufacturer's protocol. For miRNA quantitative analysis, 1 μg of RNA was reverse transcribed using the miscript system (Qiagen), and qPCR was performed with miscript miRNA primers (Qiagen). All miRNA assays were done using ssoFast Evagreen supermix (Biorad). For mRNA qPCR, total RNA was extracted using the above method, and cDNA synthesis was done with 1 μg RNA for each sample using iscript (BioRad). Six1, Smad7, and PPIB primers were part of the gene expression assay collection obtained from Applied Biosystems, and qPCR was accomplished with Roche FastStart universal real-time PCR master mix. All qPCR was performed with the BioRad CFX96. Real-time PCR arrays were acquired from SABiosciences (RT² Profiler PCR array), and included the TGF-β targets array (PAHS-235) and human stem cell array (PAHS-405). These assays were carried out following manufacturer instructions.

Western Blot Analysis.

Whole cell lysates were isolated using RIPA buffer as previously described (Ford et al., 2000). Antibodies used include: E-Cadherin (BD Biosciences), β-catenin (BD Biosciences), TβRI (SCBT), TβRII (Cell Signaling), p-Smad3 (Cell Signaling), total Smad3 (Invitrogen), Bim (Cell Signaling), p21 (Cell Signaling), β-Actin (sigma-aldrich), and β-Tubulin (Invitrogen). Cell fractionation was performed as previously described (Shutman, 2006).

Cell Adhesion Assay.

Cells were plated at a density of 1×10⁴ cells per well in 96-well plates coated with Collagen I, Collagen IV, Laminin, or Fibronectin (BD Biocoat, BD Biosciences), and assays were carried out as previously described Micalizzi et al., 2009). Absorbance was determined at 570 nm on a microplate reader (Modulus microplate, Turner Biosystems).

In Situ Hybridization and Immunohistochemistry.

Breast cancer tissue arrays were purchased from US Biomax Inc. ISH was performed with double-DIG-labeled miRNA LNA probes (Exiqon) using a modified protocol as described in (Jørgensen et al., 2010). Modification included the use of 15 μg/mL Proteinase K for 8 minutes, overnight instead of 1 hour hybridization with 40 nM of LNA probe, and a formamide containing hybridization buffer (50% Formamide, 5×SSC, 0.1% Tween, 50 μg/mL Heparin, 500 μg/mL yeast tRNA) at hybridization temperatures 30 degrees below the RNA Tm. Detection was achieved with BM purple (Roche) solution, and slides were counterstained in Nuclear Fast Red (Poly Scientific). Slides were scored on a 0-4 scale with 4 representing the most intense staining Scores were assigned independently by 3 individuals, and averaged. Serial sections of tumor arrays were previously stained and scored on a scale of 0-4 for nuclear Six1 (1:100; Atlas antibodies) and nuclear Smad3 (5 μg/mL; Zymed), using IHC protocols previously described (Micalizzi et al., 2009). The latter slides were also previous scored on the same 0-4 scale as above by a pathologist.

Luciferase Assays.

The 3′UTR of Smad7 was cloned into the psi-Check2 luciferase reporter (Promega) using XhoI and NotI sites. Cells were seeded at a density of 5×10⁴ cells per well in 24-well plates. The next day Smad7 3′UTR luciferase constructs were transfected at 50 ng per well using Lipofectamine 2000 transfection reagent (Invitrogen). Cells were harvested 48 hours after transfection, and lysates were prepared and analyzed using the dual luciferase assay following the manufacturer's protocol (Dual-Luciferase Reporter Assay System, Promega). For the TOP-flash reporter assays, cells were plated as above. The next day cells were co-transfected with 0.5 μg TOP-flash luciferase reporter and 25 ng Renilla luciferase reporter. After 48 hours, cells were harvested and analyzed in the same manner as above. All luciferase assays were analyzed on the Modulus Microplate reader (Turner Biosystems). For perfect target luciferase assays, the exact complement sequence of each miRNA was cloned into the psi-Check2 luciferase reporter as described above. Each construct was transfected into cells at 200 ng per well in a 24-well plate, media was changed next day, and lysates were prepared and isolated 48 hrs later as mentioned above.

TIC Assays.

Flow cytometry analysis and tumorshpere formation assays were performed as described previously (Farabaugh et al., 2011) For in vivo tumor initiation assays, cells were counted and serially diluted in 100 μl of 1:1 PBS/Matrigel (#354234, BD Biosciences). Diluted cells were injected underneath the nipple of the number 4 mammary fat pad of 6-week old female NOD/SCID mice. Tumor formation was monitored weekly by palpation. All animal studies were performed according to protocols reviewed and approved by the Institutional Animal Care and Use Committee at the University of Colorado Denver.

### Example 2

### Results

Six1 Regulates the miR-106b-25 Cluster of miRNAs.

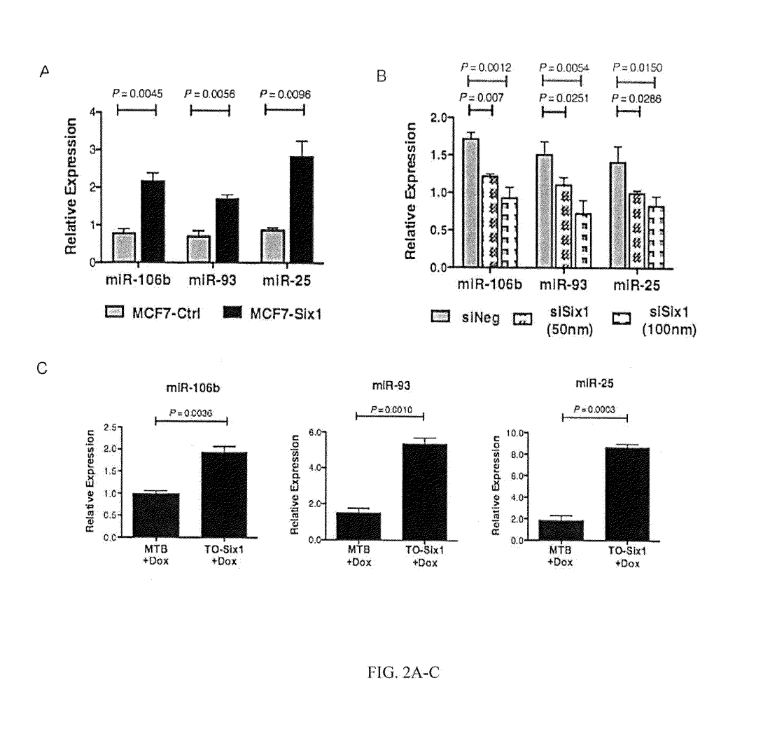

Previous studies have demonstrated substantial cross-talk between miRNAs and homeobox genes (Chopra and Mishra, 2006; Hu et al., 2010). The inventors therefore asked whether the Six1 homeoprotein might regulate miRNAs to mediate its tumorigenic and metastatic phenotypes. miRNA microarray analysis on RNA isolated from MCF7-Six1 and MCF7-Ctrl cells led to the identification of several miRNAs that were differentially expressed in a statistically significant manner between the two groups (FIG. 1A). Interestingly, the inventors identified two miRNAs, miR-106b and miR-25, that were upregulated in response to Six1 overexpression (FIG. 1A), and that belong to a cluster of miRNAs, which also includes miR-93, and reside in the 13^{th} intron of the MCM7 gene (FIG. 1B). These miRNA have previously been implicated as a pro-oncogenic cluster of miRNAs (Li et al., 2009; Poliseno et al., 2010; Fang et al., 2011). To validate the microarray results, the inventors performed quantitative real-time reverse transcriptase PCR (qRT-PCR) on an independent set of RNA isolated from MCF7-Ctrl and MCF7-Six1 cells, demonstrating that all three miRNA within the cluster are overexpressed 2-3 fold in MCF7-Six1 cells as compared to MCF7-Ctrl cells (FIG. 2A) (Ford and Pardee, 1998). In addition, siRNA knockdown of Six1 in 21PT cells (data not shown), which contain high levels of Six1 endogenously (Reichenberger et al., 2005), resulted in a clear decrease in all three miRNAs, confirming that endogenous Six1 regulates the miR-106b-25 cluster (FIG. 2B). Finally, to examine whether Six1 could regulate the miR-106b-25 cluster in vivo, the inventors analyzed expression of the miRNA cluster in transgenic mice in which Six1 was induced (using doxycline) in the mammary gland (Six1+Dox) and in control animals (Ctrl+Dox) (McCoy et al., 2009) and found that all three miRNAs, miR-106b, miR-93, and miR-25, are overexpressed in Six1 transgenic mammary glands as compared to control mammary glands (FIG. 2C). Importantly, Six1 transgenic mice develop aggressive mammary carcinomas that display multiple histological subtypes as well as an induction of an EMT (McCoy et al., 2009).

The miR-106b-25 Cluster Targets Smad7 for Repression.

It was previously shown that the miR-106b-25 cluster has the ability to overcome TGF-β mediated growth suppression via repression of the cell cycle inhibitor p21, and the pro-apoptotic factor Bim (Petrocca et al., 2008). In addition to the known role of the cluster in TGF-β growth inhibition, using target prediction analysis, the inventors found that the miR-106b-25 cluster might also play a role in activating the TGF-β pathway, providing an attractive mechanism by which Six1 could mediate the switch in TGF-β signaling from tumor suppressive to tumor promotional. Indeed, based on seed sequence alignment, the inhibitory Smad7 (I-Smad7) mRNA is a target for all three miRNAs in the cluster. Smad7 antagonizes TGF-β signaling through multiple mechanisms, including binding to TGF-β type I receptor (TβRI) and interfering with recruitment and downstream phosphorylation and activation of the receptor-Smads (R-Smads), Smad2 and Smad3. Additionally, Smad7 also functions to recruit E3 ubiquitin ligases to TβRI, resulting in its degradation (Yan et al., 2011). Therefore, repression of Smad7 by the miR-106b-25 miRNAs would be expected to activate the TGF-β signaling pathway, which is known to occur downstream of Six1 (Micalizzi et al., 2009).

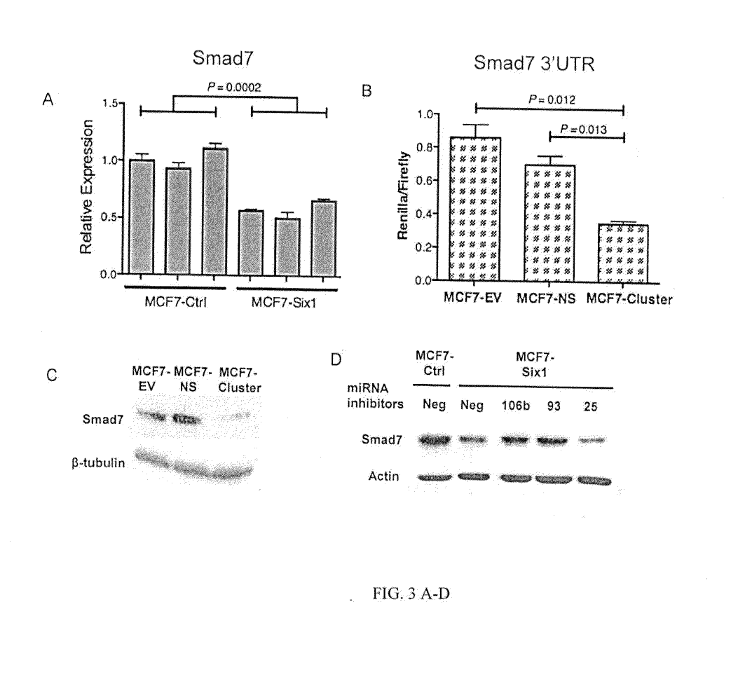

To determine if Smad7 is downregulated in response to Six1, the inventors first performed qRT-PCR on clonal isolates of MCF7-Ctrl and MCF7-Six1 cells and demonstrated that Smad7 expression is indeed reduced in MCF7-Six1 cells, where the miR-106b-25 cluster is overexpressed (FIG. 3A; FIG. 3D shows protein level differences). To further determine whether the cluster of miRNAs can directly affect Smad7 levels, the inventors generated MCF7 cell lines stably overexpressing the genomic region of the cluster (MCF7-Cluster), or control MCF7 cells expressing either the empty vector (MCF7-EV) or a non-silencing (scrambled control) vector (MCF7-NS). Importantly, stable populations expressing the cluster were chosen to overexpress each miRNA in the cluster only 2 to 3-fold, similar to what is observed with Six1 overexpression (data not shown). Transfection of a Smad7-3′UTR-luciferase construct into these cell lines demonstrates that the miR-106b-25 cluster inhibits the 3′UTR of Smad7 (FIG. 3B). Additionally, a decrease in Smad7 protein in MCF7-Cluster cells is observed when compared to MCF7-EV and MCF7-NS cells, demonstrating that a 2-3 fold increase in the miR-106b-25 cluster can downregulate endogenous Smad7 (FIG. 3C). Conversely, treatment of MCF7-Six1 cells with transient inhibitors against the individual miRNAs leads to a de-repression of Smad7 protein, with miR-106b and miR-93 being the major mediators of this effect (FIG. 3D). Efficacy of the miRNA inhibitors is demonstrated by relative activity of luciferase reporters, containing target sites for each miRNA, in the inhibitor transfected cells (data not shown).

The miR-106b-25 Cluster Activates TGFβ Signaling.

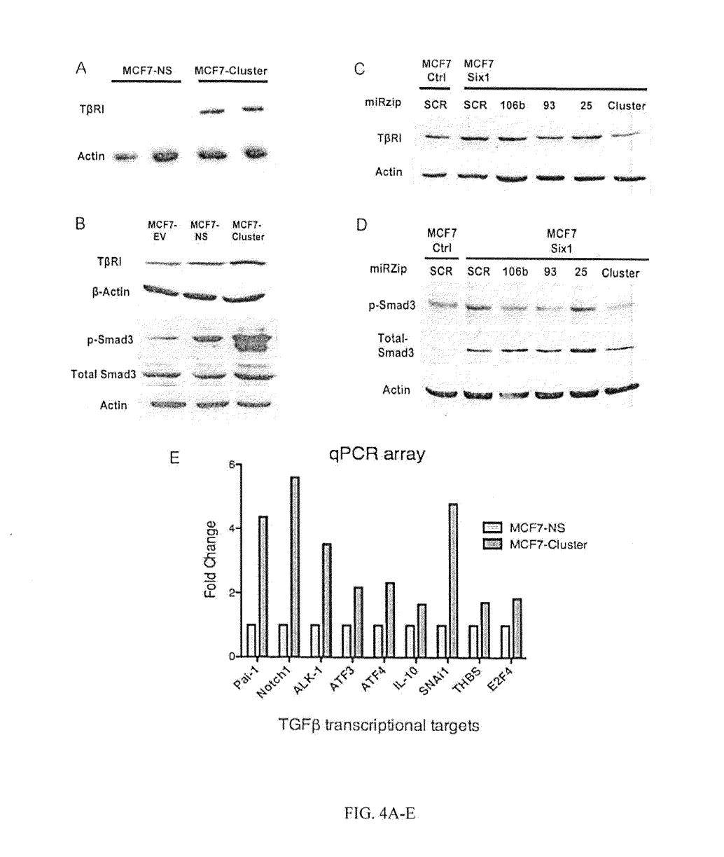

Because the miR-106b-25 cluster represses Smad7 in MCF7 cells, and because Six1 overexpression, which leads to increased levels of the miR-106b-25 miRNAs, activates TGF-β signaling (Micalizzi et al., 2009), the inventors asked whether this cluster of miRNAs, which is known to overcome TGF-β-mediated growth inhibition, is also sufficient to activate the TGF-β pathway. Indeed, with both transient and stable overexpression of the miR-106b-25 cluster, the inventors observed an increase in TβRI protein levels (FIG. 4A and FIG. 4B), as well as an increase in activated TGF-β signaling as measured by p-Smad3 levels (FIG. 4B). To further determine if the miR-106b-25 miRNAs are necessary for the previously observed induction of TGF-β signaling by Six1, the inventors utilized a stable lentiviral miRNA knockdown system (miRZip) to inhibit the miRNAs within the cluster either individually or together. Efficacy of the miRZips was demonstrated by examining their effects on endogenous targets of the miR-106b-25 cluster, p21 and BIM (data not shown). Inhibition of miR-93, as well as the entire cluster in MCF7-Six1 cells reverses the Six1-induced increase in TβRI (FIG. 4C), and inhibition of miR-106b, miR-93, as well as the entire cluster reverses the Six1-induced increase in p-Smad3 (FIG. 4D).

Interestingly, previous reports have identified the miR-106b-25 cluster miRNAs as targeting the TGF-β type II receptor (TBRII), resulting in repression of this protein. Analysis of TβRII protein in the inventors' MCF7-Cluster cells did not show a repression of TβRII protein as compared to MCF7-NS cells (data not shown). Similarly, the inventors also did not observe significant downregulation of TβRII protein levels in MCF7-Six1 versus MCF7-Ctrl cells (data not shown). Thus, 2 to 3-fold overexpression of the miR-106b-25 cluster in MCF7 cells leads primarily to alterations in the TGF-β pathway that would be expected to be activating, as opposed to inactivating.

To analyze global changes in TGF-β signaling, the inventors performed microarray analysis on MCF7-NS versus MCF7-Cluster cells to examine whether the presence of the miR-106b-25 cluster alters the TGF-β response signature (TβRS) (Padua et al., 2008), similar to what is observed with Six1 overexpression (Micalizzi et al., 2009). Hierarchical clustering confirmed differential regulation of many of the genes in the TβRS between MCF7-NS and MCF7-Cluster cells, demonstrating that TGF-β signaling is indeed altered in response to miR-106b-25 overexpression (data not shown). In addition, the inventors performed a qRT-PCR array to examine alterations in expression of TGF-β target genes using MCF7-NS and MCF7-Cluster cells, demonstrating that TGF-β signaling is clearly activated downstream of the miR-106b-25 cluster, as numerous TGF-β transcriptional targets are upregulated in MCF7 cells overexpressing the cluster (FIG. 4E). Of the 84 genes responsive to TGF-β signaling on the array, 47 were upregulated 1.5-fold or more in MCF7-Cluster cells, 8 genes were downregulated, and the rest remained unchanged (data not shown). Together, these data demonstrate that the miR-106b-25 cluster is capable of activating TGF-β signaling in breast cancer cells, and suggest that this one cluster, which can also overcome the growth suppressive effects of TGF-β, may be responsible for the switch in TGF-β signaling from tumor suppressive to tumor promotional.

The miR-106b-25 Cluster Mediates Some Features of EMT.

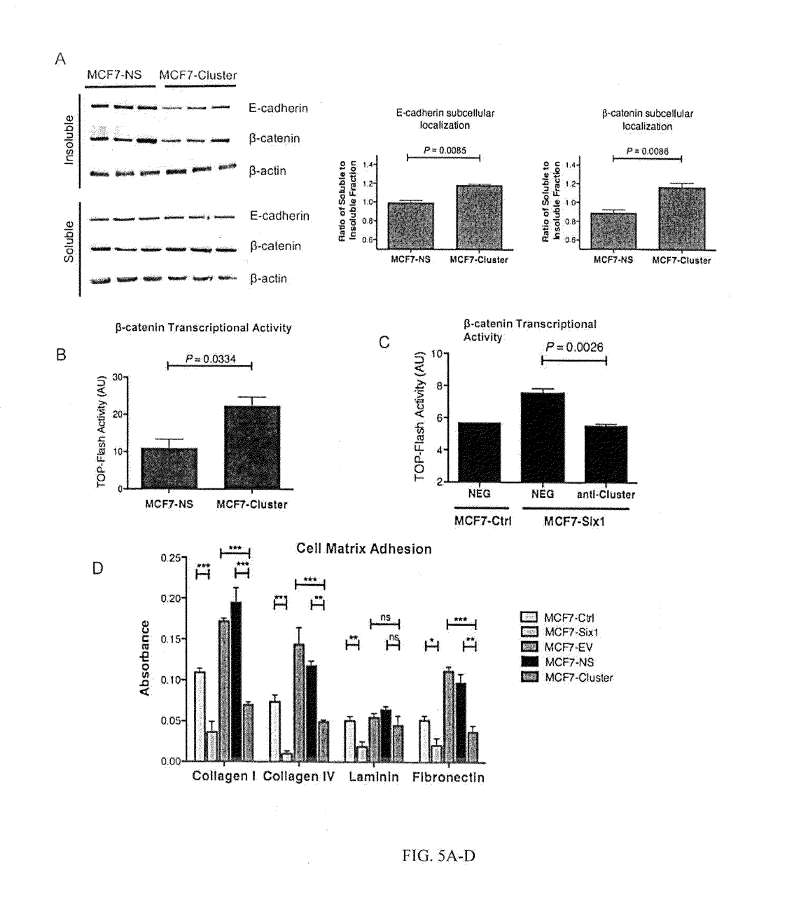

The inventors previously reported that Six1 overexpression leads to an induction of EMT, which is dependent on TGF-β signaling (Micalizzi et al., 2009). Because the miR-106b-25 cluster is sufficient to activate TGF-β signaling, they asked if this cluster is sufficient to mediate phenotypes associated with EMT. One of the hallmarks of EMT is the loss of membranous E-cadherin from the adherens junctions. The inventors thus analyzed the subcellular localization of E-cadherin from each cell line and demonstrated that E-cadherin is indeed decreased in the insoluble, or membrane bound, fraction of the cell in response to miR-106b-25 overexpression (FIG. 5A). β-catenin, which is normally in the membranous adherens junctions with E-cadherin, is also decreased in the insoluble fraction of MCF7-Cluster cells (FIG. 5A). Because redistribution of β-catenin away from the membrane may result in increased nuclear localization and subsequent transcriptional activation, the inventors measured β-catenin transcriptional activity using the TOP-flash luciferase reporter. Concomitant with the loss of β-catenin from the membrane, MCF7-Cluster cells also exhibit an increase in TOP-flash reporter activity over MCF7-NS cells, similar to the phenotype observed with Six1 overexpression (FIG. 5B). To determine if the miR-106b-25 miRNAs are necessary for the Six1-induced increases in β-catenin transcriptional activation, the inventors treated MCF7-Six1 cells with inhibitors toward all three miRNA (miR-106b, miR-93 and miR-25) together and measured TOP-flash activity. A repression of TOP-flash activity in this context demonstrates that Six1 is dependent on the miR-106b-25 miRNAs to induce β-catenin transcriptional activity (FIG. 5C). Finally, the inventors observed a decrease in cell-matrix adhesion to Collagen I, Collagen IV, and Fibronectin in the MCF7-Cluster cells as compared to the MCF7-NS and MCF7-EV cells, similar to the decrease in adhesion observed with Six1 overexpression in MCF7 cells (FIG. 5D).

The miR-106b-25 Cluster Increases Tumor Initiating Cell Characteristics.

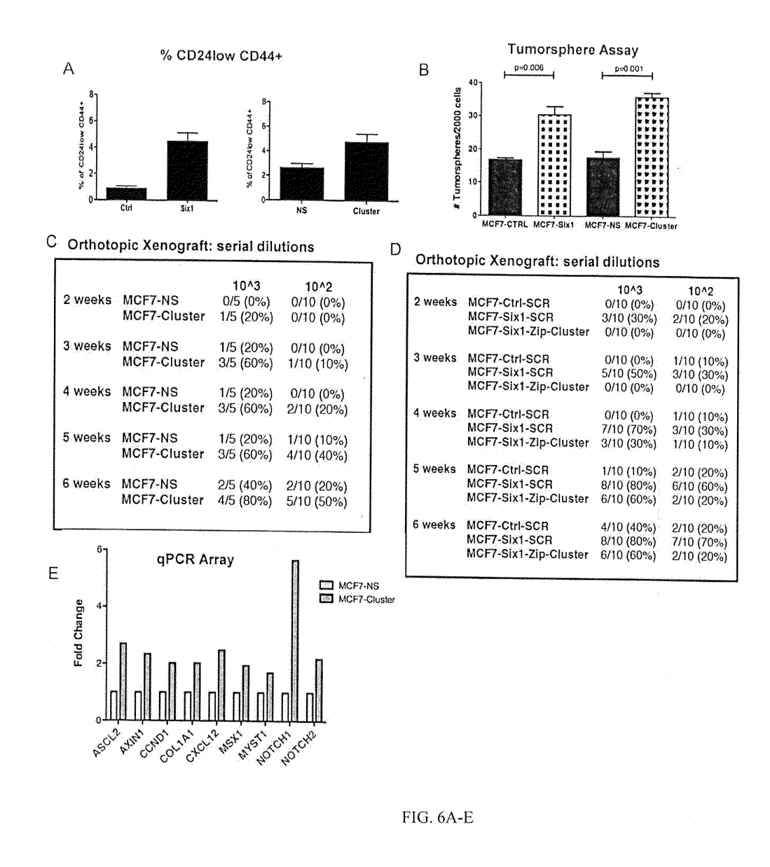

Many genes that induce EMT-like phenotypes also induce tumor initiating cell (TIC) phenotypes (Mani et al., 2008). Indeed, the inventors recently demonstrated that Six1 induces a TIC phenotype in both a transgenic mouse model (McCoy et al., 2009) and when overexpressed in MCF7 cells (Farabaugh et al., 2011). To determine if the miR-106b-25 miRNAs, which can induce properties of EMT, can also induce TIC characteristics, the inventors performed flow cytometry for the cell surface TIC-associated markers CD24 and CD44 (Al-Hajj et al., 2003), and found that miR-106b-25 did indeed increase the percentage of CD24low CD44+ cells, similar to Six1 (FIG. 6A). Additionally, secondary tumorsphere assays performed with MCF7-Cluster and MCF7-NS cells demonstrated that, similar to MCF7-Six1 cells, MCF7-Cluster cells could increase tumorsphere formation, a measure of functional TICs within a population (FIG. 6B). To further test for functional TIC characteristics using an in vivo assay, the inventors injected cells at limiting dilutions into the mammary fat pad of NOD-SCID mice. MCF7-Cluster cells were able to initiate tumors with a greater frequency then MCF7-NS cells, both when 1000 and 100 cells were injected (FIG. 6C). In order to determine if the miR-106b-25 cluster was necessary for Six1-induced increases in TICs in vivo, the inventors utilized their MCF7-Six1-miRZip-Cluster cells (in which all three miRNAs are inhibited), and transplanted these cells into the mammary gland at limiting dilutions, along with miRZip-SCR controls in both MCF7-Ctrl and MCF7-Six1 cells. Inhibition of the miR-106b-25 miRNAs in MCF7-Six1 cells demonstrates a reduction in tumor initiating ability back to the TIC frequency seen in MCF7-Ctrl cells (FIG. 6D). Lastly, the inventors performed a human stem cell qRT-PCR array containing genes related to the identification, growth, and differentiation of stem cells. Of the 84 genes on this array, 53 were upregulated more than 1.5-fold in MCF7-Cluster cells (FIG. 6E). Together these data demonstrate for the first time a role for the miR-106b-25 cluster in both EMT and in increased TIC capacity.

The miR-106b-25 Cluster Correlates with Six1 and Activated TGF-β Signaling in Human Breast Cancer.

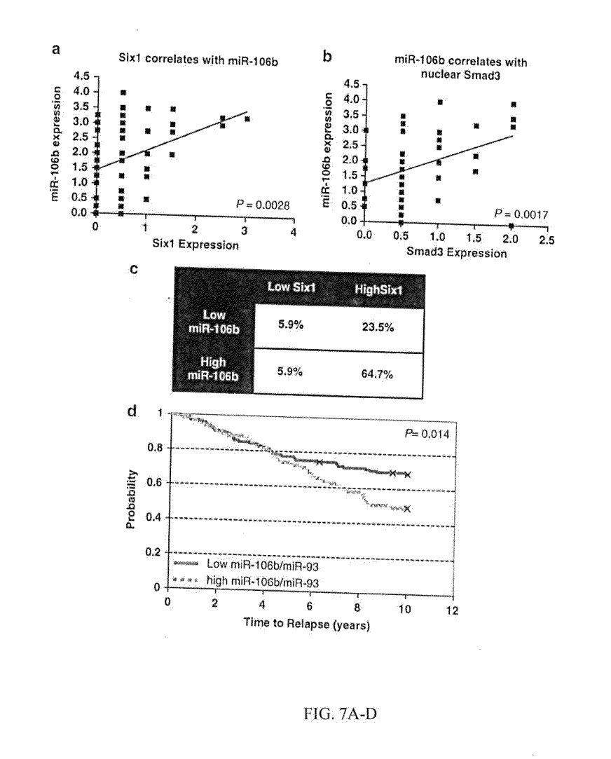

To determine if the Six1/miR-106b-25/activated TGF-β signaling axis is relevant to human breast cancer, the inventors obtained human breast cancer tissue arrays containing 71 cases of invasive ductal carcinoma, which matched cases on which they had previously performed immunohistochemistry (IHC) for nuclear Six1 and nuclear Smad3 (as an indicator of activated TGF-β signaling) (Micalizzi et al., 2009). The inventors then performed in situ hybridization (ISH) for miR-106b (as a representative of the miR-106b-25 cluster), to compare expression of this cluster family member with Six1 and activated TGF-β signaling. Importantly, miR-106b and Six1 expression significantly correlate in breast cancer tissues (p=0.0028, Spearman R=0.3927) (FIG. 7A), as do miR-106b and nuclear Smad3 (p=0.0017, Spearman R=0.3972) (FIG. 7B). In addition, the greatest percentage of tumors exhibited activated TGF-β signaling when both miR-106b and Six1 were highly expressed (64.7% show increased nuclear Smad3 when both miR-106b and Six1 are high) (FIG. 7C). Together, these data strongly suggest a critical role for miR106b-25 in the Six1-induced activation of TGF-β signaling in human breast cancer. To explore the prognostic value of these miRNA in human breast cancers, the inventors examined a publicly available dataset comprised of miRNA expression in early-invasive breast cancers (Buffa et al., 2011). FIG. 7D demonstrates that patients whose tumors express high miR-106b and high miR-93 together have a significantly shortened time to relapse. Analysis of individual miRNA expression in these tumors also demonstrates a significant correlation with high miR-93 (data not shown), as well as a trend toward shortened time to relapse with high miR-106b and high expression of all three miRNA (data not shown). However, miR-25 expression does not demonstrate any difference in patient outcome (data not shown), further suggesting that miR-106b and miR-93 are the primary regulators of this response.

### Example 3

### Discussion

Previous research has highlighted the importance of Six1 in breast cancer progression and metastasis. Central to this process is the role of TGF-β signaling, as Six1-induced EMT, TIC, and late stage metastasis are all dependent on an upregulation of this pathway (Micalizzi et al., 2009). Interestingly, Six1 not only activates TGF-β signaling, but it can also switch TGF-β signaling from tumor suppressive to tumor promotional (Micalizzi et al., 2010), a phenomenon that is not well understood and that is of considerable import in cancer pathogenesis (Inman, 2011). Work described herein implicates miRNAs in this process.

In the present study, the inventors identify a cluster of miRNA, the miR-106b-25 cluster, as a target of Six1. These miRNAs have previously been shown to overcome TGF-β mediated growth inhibition, through repression of p21 and BIM (Petrocca et al., 2008). While some cancers display mutations in the core components of the TGF-β pathway (Blackford et al., 2009), breast cancers typically retain functional TGF-β signaling and instead selectively inhibit the tumor suppressive arm of TGF-β (Padua and Massagué, 2009). The data herein not only provide a mechanism for how Six1 may silence TGF-β-mediated growth inhibition in breast cancers, but also for how Six1 activates the TGF-β pathway. In fact, to the inventors' knowledge, this is the first demonstration that the same miRNA cluster that overcomes TGF-b mediated growth suppression can in fact also promote TGF-β signaling.

Indeed, the inventors show that miR-106b-25 miRNAs can target the TGF-β inhibitor Smad7, and that upregulation of miR106b-25 leads to an increase in TβRI. This increase in TbRI likely occurs, at least in part, due to Smad7 downregulation, since Smad7 is known to mediate degradation of the TβRI protein (Yan et al., 2009). Recently, the inventors demonstrated that upregulation of TβRI protein is necessary and sufficient for TGF-β activation and the induction of EMT downstream of Six1 in MCF7 cells (Micalizzi et al., 2010). Because they have also shown that Six1 is able to transcriptionally activate TβRI (Micalizzi et al., 2010), these data demonstrate that Six1 impinges on TGF-β signaling via multiple mechanisms that converge on TβRI, including both transcriptional and post-transcriptional mechanisms. Consistent with previous data demonstrating that TβRI overexpression is sufficient for TGF-β pathway activation, the inventors also observe an activation of this pathway with overexpression of the miR-106b-25 miRNAs. The transcriptional targets of the TGF-β signaling pathway that are altered by miR-106b-25 include genes involved in differentiation (Snail), proliferation and migration (ALK-1, ATF3, IL-10, Pai-1), anti-apoptosis (THBS1), as well as genes involved in transcriptional reguation (E2F4, ATF3, ATF4) (FIG. 4E). Other genes upregulated by miR-106b-25, such as Notch1, are known to be involved in TGF-β mediated EMT (Zavadil and Böttinger, 2005).

Several lines of evidence have demonstrated the miR-106b-25 cluster and its individual miRNAs have pro-oncogenic functions, including mediating pro-proliferative and anti-apoptotic phenotypes (Li et al., 2009; Kan et al., 2009). The results of this study expand the oncogenic potential of this miRNA cluster by demonstrating for the first time that these miRNA can also induce properties of EMT and tumor initiating cell characteristics. The EMT changes induced by these miRNA are consistent with the oncogenic EMT phenotype induced by Six1 in MCF7 cells (FIGS. 5A-C), suggesting that overexpression of the miR-106b-25 miRNAs may partly contribute to the induction of EMT downstream of Six1.

It is well recognized that the induction of EMT leads to an increase in stem/progenitor cell properties (Mani et al., 2008). Indeed, Six1 transgenic mice whose mammany tumors display features of EMT, also demonstrate an increase in the stem/progenitor cell population as well as increased mammosphere-forming ability in their mammary epithelial cells (McCoy et al., 2009). Increased expression of the miR-106b-25 miRNAs in the mammary glands of Six1 transgenic mice suggests a possible role for these miRNA in regulation of the stem/progenitor pool (FIG. 2C). Indeed, these results show for the first time that the miR-106b-25 miRNAs are sufficient to increase TIC capacity, and that they are required for the ability of Six1 to induce TIC characteristics in vivo. Interestingly, many recent studies have implicated the miR-106b-25 cluster in stem/progenitor cell biology (Brett et al., 2011; Qian et al., 2008; Ho et al., 2011). Of interest, it was recently demonstrated that miR-106b and miR-93 can enhance reprogramming of somatic cells into induced pluripotent stem cells (iPSCs) along with expression of iPSC transcription factors (Li et al., 2011). The iPSC phenotype, however, is dependent on downregulation of TGF-β signaling, where the miRNAs target TβRII, while the TIC phenotype is known to be associated with an upregulation of TGF-β signaling (Mani et al., 2008). In this study, the inventors did not observe a downregulation of TβRII in response to Six1 or miR-106b-25 overexpression in MCF7 cells (Micalizzi et al., 2010). However, interestingly, both the iPSC data and these data suggest a role for the miR-106b-25 cluster in the induction of stem cell properties, possibly through their ability to regulate TGF-β signaling in a context dependent, and seemingly opposite, manner.

Lastly, these data demonstrate a significant correlation between miR-106b, Six1, and activated TGF-β signaling (nuclear Smad3) in human breast cancers. Critically, the inventors show that tumors that express both high Six1 and high miR-106b have the highest percentage of activated TGF-β signaling (64.7%) (FIGS. 7A-C). Of note, the data also show that in the presence of high Six1 while miR-106b expression is low, 23.5% of tumors have activated TGF-β signaling, as opposed to 5.9% in tumors with low levels of Six1. These data suggest again that Six1 may activate TGF-β signaling through multiple mechanisms, however the marked increase in TGF-β signaling when Six1 and miR-106b are both highly expressed strongly suggests that the Six1/miR-106b-25 axis is critical for activation of TGF-β signaling in human breast cancer.

In closing, these data have important ramifications for breast cancer treatment. Due to the traditional difficulties in targeting transcription factors such as Six1, and the increasing promise for miRNAs as therapeutic targets (Nana-Sinkam and Croce, 2011), the miR-106b-25 cluster could prove to be an effective target in cancers that express high levels of Six1. Furthermore, TGF-β inhibitors are currently in clinical trials. Since TGF-β signaling can be tumor suppressive or tumor promotional, depending on context, one of the greatest concerns surrounding the use of TGF-β inhibitors in cancer is how to predict which patients will benefit. These studies suggest a mechanism that the inventors hypothesize provides a novel molecular explanation for the TGF-β paradox in breast cancers and may help to resolve this clinical conundrum. Namely, examining breast tumors for Six1 and/or miR-106b-25 expression may ultimately provide a means to distinguish patients likely to benefit from TGF-β inhibitors from those who may actually be harmed by such treatments.

All of the methods and apparatus disclosed and claimed herein can be made and executed without undue experimentation in light of the present disclosure. While the compositions and methods of this invention have been described in terms of particular embodiments, it will be apparent to those of skill in the art that variations may be applied to the methods and apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VI. REFERENCES

The following references, to the extent that they provide exemplary procedural or other details supplementary to those set forth herein, are specifically incorporated herein by reference.
U.S. Pat. No. 4,196,265
U.S. Pat. No. 4,415,723
U.S. Pat. No. 4,458,066
U.S. Pat. No. 4,683,195
U.S. Pat. No. 4,683,202
U.S. Pat. No. 4,800,159
U.S. Pat. No. 4,816,567
U.S. Pat. No. 4,867,973
U.S. Pat. No. 4,883,750
U.S. Pat. No. 4,981,957
U.S. Pat. No. 5,118,800
U.S. Pat. No. 5,143,854
U.S. Pat. No. 5,202,231
U.S. Pat. No. 5,242,974
U.S. Pat. No. 5,279,721
U.S. Pat. No. 5,288,644
U.S. Pat. No. 5,319,080
U.S. Pat. No. 5,324,633
U.S. Pat. No. 5,359,044
U.S. Pat. No. 5,384,261
U.S. Pat. No. 5,393,878
U.S. Pat. No. 5,405,783
U.S. Pat. No. 5,412,087
U.S. Pat. No. 5,424,186
U.S. Pat. No. 5,429,807
U.S. Pat. No. 5,432,049
U.S. Pat. No. 5,436,327
U.S. Pat. No. 5,445,934
U.S. Pat. No. 5,446,137
U.S. Pat. No. 5,466,786
U.S. Pat. No. 5,468,613
U.S. Pat. No. 5,470,710
U.S. Pat. No. 5,472,672
U.S. Pat. No. 5,492,806
U.S. Pat. No. 5,503,980
U.S. Pat. No. 5,510,270
U.S. Pat. No. 5,514,785
U.S. Pat. No. 5,519,134
U.S. Pat. No. 5,525,464
U.S. Pat. No. 5,527,681
U.S. Pat. No. 5,529,756
U.S. Pat. No. 5,532,128
U.S. Pat. No. 5,545,531
U.S. Pat. No. 5,547,839
U.S. Pat. No. 5,554,501
U.S. Pat. No. 5,556,752
U.S. Pat. No. 5,561,071
U.S. Pat. No. 5,565,332
U.S. Pat. No. 5,567,811
U.S. Pat. No. 5,571,639
U.S. Pat. No. 5,576,427
U.S. Pat. No. 5,580,726
U.S. Pat. No. 5,580,732
U.S. Pat. No. 5,591,722
U.S. Pat. No. 5,593,839
U.S. Pat. No. 5,597,909
U.S. Pat. No. 5,599,672
U.S. Pat. No. 5,599,695
U.S. Pat. No. 5,610,287
U.S. Pat. No. 5,610,300
U.S. Pat. No. 5,624,711
U.S. Pat. No. 5,627,053
U.S. Pat. No. 5,631,134
U.S. Pat. No. 5,639,603
U.S. Pat. No. 5,639,873
U.S. Pat. No. 5,646,265
U.S. Pat. No. 5,654,413
U.S. Pat. No. 5,658,734
U.S. Pat. No. 5,658,873
U.S. Pat. No. 5,661,028
U.S. Pat. No. 5,665,547
U.S. Pat. No. 5,667,972
U.S. Pat. No. 5,670,633
U.S. Pat. No. 5,695,940
U.S. Pat. No. 5,700,637
U.S. Pat. No. 5,700,920
U.S. Pat. No. 5,739,169
U.S. Pat. No. 5,744,305
U.S. Pat. No. 5,792,747
U.S. Pat. No. 5,795,715
U.S. Pat. No. 5,800,992
U.S. Pat. No. 5,801,005
U.S. Pat. No. 5,807,522
U.S. Pat. No. 5,824,311
U.S. Pat. No. 5,830,645
U.S. Pat. No. 5,830,880
U.S. Pat. No. 5,837,196
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,846,945
U.S. Pat. No. 5,847,219
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,871,928
U.S. Pat. No. 5,876,932
U.S. Pat. No. 5,882,864
U.S. Pat. No. 5,889,136
U.S. Pat. No. 5,900,481
U.S. Pat. No. 5,902,880
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
U.S. Pat. No. 6,004,755
U.S. Pat. No. 6,087,102
U.S. Pat. No. 6,146,886
U.S. Pat. No. 6,268,490
U.S. Pat. No. 6,368,799
U.S. Pat. No. 6,383,749
U.S. Pat. No. 6,395,713
U.S. Pat. No. 6,525,191
U.S. Pat. No. 6,531,584
U.S. Pat. No. 6,600,032
U.S. Pat. No. 6,617,112
U.S. Pat. No. 6,638,717
U.S. Pat. No. 6,670,461
U.S. Pat. No. 6,720,138
U.S. Pat. No. 6,770,748
U.S. Pat. No. 6,794,499
U.S. Pat. No. 7,034,133
U.S. Pat. No. 7,053,207
U.S. Patent Publn. 2008/0009439
U.S. Patent Pubn. 2003/0082807
U.S. Patent Pubn. 2003/0207841
U.S. Patent Pubn. 2004/0014959
U.S. Patent Pubn. 2004/0143114
U.S. Patent Pubn. 2004/0171570
U.S. Patent Pubn. 2004/0219565
U.S. Patent Pubn. 2007/0213292
U.S. Patent Pubn. 2007/0287831
U.S. Patent Pubn. 2008/0015162
U.S. Patent Pubn. 2008/0032945
Akhtar et al., Trends in Cell Bio., 2:139, 1992.
Akhtar, In: Delivery Strategies for Antisense Oligonucleotide Therapeutics, 1995.
Akinc et al. Nat. Biotechnol., 2008 May; 26(5):561-9, 2008.
Al-Hajj et al., Proc. Natl. Acad. Sci. USA, 100(7):3983-3988, 2003.
Allerson et al., J. Med. Chem., 48:901-904, 2005.
Ambros et al. RNA, 9:277-279, 2003.
Ando et al., Febs J., 272(12):3026-3041, 2005.
Antibodies: A Laboratory Manual, Cold Spring Harbor Laboratory, 1988.
Arap et al., Cancer Res., 55(6):1351-1354, 1995.
Austin-Ward and Villaseca, Revista Medica de Chile, 126(7):838-845, 1998.
Bakhshi et al., Cell, 41(3):899-906, 1985.
Bellus, J. Macromol. Sci. Pure Appl. Chem., A31(1): 1355-1376, 1994.
Bentin and Nielsen, Biochemistry, 35:8863-8869, 1996.
Berger et al., Cell, 133(7):1266-1276, 2008.
Blackford et al., Clinical Cancer Res., 15(14):4674-4679, 2009.
Bosher and Labouesse, Nat. Cell. Biol., 2(2):E31-E36, 2000.
Braasch et al., Methods, 23(2):97-107, 2001.
Brennecke et al., Cell, 113(1):25-36, 2003.
Brett et al., Aging (Albany N.Y.), 3(2):108-124, 2011.
Buffa et al., Cancer Res., 71: 5635-5645, 2011.
Bukowski et al., Clinical Cancer Res., 4(10):2337-2347, 1998.
Caldas et al., Cancer Res., 54:3568-3573, 1994.
Calin et al., Proc. Natl. Acad. Sci. USA, 99(24):15524-15529, 2002.
Campbell, In: Monoclonal Antibody Technology, Laboratory Techniques in Biochemistry and Molecular Biology, Burden and Von Knippenberg (Eds.), Elseview, Amsterdam, 13:71-74/75-83, 1984.
Caplen et al., Gene, 252(1-2):95-105, 2000.
Carrington and Ambros, Science, 301(5631):336-338, 2003.
Chang et al., J. Exp. Med., 200:1359-1370, 2004.
Chen et al., Fed. of Eur. Biochem Soc., 309:115-118, 1992.
Chen et al., Mol. Microbiol., 53843-856, 2004.
Cheng et al., Cancer Res., 54(21):5547-5551, 1994.
Chopra and Mishra, Bioessays, 28(5):445-448, 2006.
Choung et al., Biochem. Biophys. Res. Commun., 342:919-927, 2006.
Chowrira et al., J. Biol. Chem., 269(41):25856-25864, 1994.
Christodoulides et al., Microbiology, 144(Pt 11):3027-3037, 1998.
Cleary and Sklar, Proc. Natl. Acad. Sci. USA, 82(21):7439-7443, 1985.
Cleary et al., J. Exp. Med., 164(1):315-320, 1986.
Coletta et al., Proc. Natl. Acad. Sci. USA, 101(17):6478-6483, 2004.
Couture et al., Trends in Genetics, 12:510, 1996.
Davidson et al., J. Immunother., 21(5):389-398, 1998.
Demidov et al., ChemBiochem., 2:133-139, 2001.
Egholm et al., Nature, 365(6446):566-568, 1993.
Elayadi et al., Nucleic Acids Res., 29(8):1683-9, 2001.
Elbashir et al., Genes Dev., 5(2):188-200, 2001.
Elmén et al., Nature, 452(7189):896-9, 2008.
Esau et al. Cell Metab., 3(2):87-98, 2006.
European Appln. 320 308
European Appln. 329 822
European Appln. EP 373 203
European Appln. EP 785 280
European Appln. EP 799 897
Fang et al., Oncogene, 30(7):806-821, 2011.
Farabaugh et al., Oncogene, 1-11, 2011.
Faruqi et al., Proc. Natl. Acad. Sci. USA, 95:1398-1403, 1998.
Fatkin et al., J. Clin. Invest., 106(11):1351-1359, 2000.
Fire et al., Nature, 391(6669):806-811, 1998.
Ford and Pardee, Proc. Natl. Acad. Sci. USA, 1-6, 1998.
Ford et al., J. Biol. Chem., 275(29):22245-22254, 2000.
Fridlender et al., Cancer Cell. 16(3):183-94, 2009.
Frieden et al., Nucl. Nucleic Acids, 22:1041-1043, 2003.
Frohman, In: PCR Protocols: A Guide To Methods And Applications, Academic Press, N.Y., 1990.
GB Appln. 2 202 328
Gefter et al., Somatic Cell Genet., 3:231-236, 1977.
Goding, In: Monoclonal Antibodies: Principles and Practice, 2d ed., Academic Press, Orlando, Fl, pp 60-61, 71-74, 1986.
Grishok et al., Science, 287:2494-2497, 2000.
Haeberli et al., Nucleic Acids Res., 33:3965-3975, 2005.
Hall et al., Nucleic Acids Res., 32:5991-6000, 2004.
Hall et al., Nucleic Acids Res., 34:2773-2781, 2006.
Hanibuchi et al., Int. J. Cancer, 78(4):480-485, 1998.
Hassani et al., J. Gene Med., 7(2):198-207, 2005.
Hellstrand et al., Acta Oncologica, 37(4):347-353, 1998.
Ho et al., J. Am. Soc. Nephrol., 22(6):1053-1063, 2011.
Hofland and Huang, Handb. Exp. Pharmacol., 137:165, 1999.
Holstege et al., EMBO J., 16:7468-7480, 1997.
Hoshika et al., Nucleic Acids Res., 32:3815-3825, 2004.
Hu et al., Nucleic Acids Res., 38(16):5472-5478, 2010.
Hui and Hashimoto, Infection Immun., 66(11):5329-5336, 1998.
Hussussian et al., Nat. Genet., 8(1):15-21, 1994.
Hutvagner et al., PLoS Biol., 2(4):E98, 2004.
Inman, Curr. Opin. Genet. Dev., 21(1):93-99, 2011.
Innis et al., Proc. Natl. Acad. Sci. USA, 85(24):9436-9440, 1988.
Izant and Weintraub, Science, 229:345, 1985.
Jorgensen et al., Methods, 52(4):375-381, 2010.
Ju et al., Gene Ther., 7(19):1672-1679, 2000.
Kahl et al., J. Mol. Biol., 299:75-89, 2000.
Kaihatsu et al., Biochem., 42(47):13996-4003, 2003.
Kaihatsu et al., Biochemistry, 43(45):14340-7, 2004.
Kaihatsu et al., Chem. Biol., 11:749-758, 2004.
Kamb et al., Nat. Genet., 8(1):23-26, 1994.
Kamb et al., Science, 2674:436-440, 1994.
Kan et al., Gastroenterology, 136(5):1689-1700, 2009.
Kashani-Sabet et al., Antisense Res. Dev., 2:3, 1992.
Kaur et al., Biochemistry, 45(23):7347-7355, 2006.
Kawakami et al., Bioessays, 22(7):616-626, 2000.
Kawakami et al., Nucleic Acids Res., 24(2):303-310, 1996.
Kelly & Morris, J. Immunotoxicol. 7(1):15-26, 2010.
Kerr et al., Br. J. Cancer, 26(4):239-257, 1972.
Ketting et al., Cell, 99(2):133-141, 1999.
Kiriazis and Kranias, Annu. Rev. Physiol., 62:321-351, 2000.
Kohler and Milstein, Eur. J. Immunol., 6:511-519, 1976.
Kohler and Milstein, Nature, 256:495-497, 1975.
Koshkin et al., Bioorg. Med. Chem. Lett., 8(16):2219-22, 1998.
Krenz and Robbins, J. Am. Coll. Cardiol., 44:2390-2397, 2004.
Krützfeldt et al., Nature, 438:685-689, 2005.
Kumar et al., Biochem. Pharmacol., 55:775-783, 1998.
Kwoh et al., Proc. Natl. Acad. Sci. USA, 86:1173, 1989.
Lagos-Quintana et al., Science, 294(5543):853-858, 2001.
Laping et al., Mol. Pharmacol. 62(1):58-64), 2002.
Larsen and Nielsen, Nucl. Acids Res., 24:458-463, 1996.
Lau et al., Science, 294(5543):858-862, 2001.
Lee and Ambros, Science, 294(5543):862-864, 2001.
Lee et al., ACS Symp. Ser., 752:184, 2000.
Li et al., Nature, 426(6964):247-254, 2003.
Li et al., Cancer Sci., 100(7):1234-1242, 2009.
Li et al., EMBO J., 30(5):823-834, 2011.
Li et al., Proc. Natl. Acad. Sci. USA, 102:19231-19236, 2005.
Lin and Avery, Nature, 402:128-129, 1999.
Lowes et al., J. Clin. Invest., 100(9):2315-2324, 1997.
Mani et al., Cell, 133(4):704-715, 2008.
Maurer et al., Mol. Membr. Biol., 16:129, 1999.
McCoy et al., J. Clin. Invest., 119(9):2663-2677, 2009.
McGarry and Lindquist, Proc. Natl. Acad. Sci. USA, 83:399, 1986.
McMahon et al., Life Sci., 71(3):325-3, 2002.
Meister et al., Mol. Cell., 15:185-197, 2004.
Micalizzi et al., Cancer Res., 70(24):10371-10380, 2010.
Micalizzi et al., J. Clin. Invest., 119(9):2678-2690, 2009.
Mitchell et al., Ann. NY Acad. Sci., 690:153-166, 1993.
Mitchell et al., J. Clin. Oncol., 8(5):856-869, 1990.
Miyata et al., Circ. Res., 86(4):386-390, 2000.
Mollegaard et al., Proc. Natl. Acad. Sci. USA, 91:3892-3895, 1994.
Montgomery et al., Proc. Natl. Acad. Sci. USA, 95:15502-15507, 1998.
Mori et al., Cancer Res., 54(13):3396-3397, 1994.
Morita et al., Bioorg. Med. Chem., 11(10):2211-26, 2003.
Morris et al., Science, 305:1289-1292, 2004.
Morton et al., Arch. Surg., 127:392-399, 1992.
Nana-Sinkam and Croce, Transl. Res., 157(4):216-225, 2011.
Nielsen et al., Science, 254:1497-1500, 1991.
Nobori et al., Nature (London), 368:753-756, 1995.
Noyes et al., Cell, 133(7):1277-1289, 2008.
Ohara et al., Proc. Natl. Acad. Sci. USA, 86:5673-5677, 1989.
Ohkawa et al., Nucleic Acids Symp. Ser., 27:156, 1992.
Ojwang et al., Proc. Natl. Acad. Sci. USA, 89:10802, 1992.
Okamoto et al., Proc. Natl. Acad. Sci. USA, 91(23):11045-11049, 1994.
Oliver et al., Development, 121:4045-4055, 1995.
Orlow et al., Cancer Res, 54(11):2848-2851, 1994.
Orlow et al., Int. J. Oncol., 15(1):17-24, 1994.
Padua and Massagué, Cell Res., 19(1):89-102, 2009.
Padua et al., Cell, 133(1):66-77, 2008.
Paroo and Corey, Trends Biotechnol., 22(8):390-4, 2004.
Pasquinelli et al., Ann. Rev. Cell Dev. Biol., 18:495-513, 2002.
PCT Appln. PCT/US87/00880
PCT Appln. PCT/US89/01025
PCT Appln. WO 00/44914
PCT Appln. WO 00/53722
PCT Appln. WO 01/36646
PCT Appln. WO 01/38580
PCT Appln. WO 01/68255
PCT Appln. WO 01/68836
PCT Appln. WO 03/020898
PCT Appln. WO 03/022421
PCT Appln. WO 03/023058
PCT Appln. WO 03/029485
PCT Appln. WO 03/040410
PCT Appln. WO 03/053586
PCT Appln. WO 03/066906
PCT Appln. WO 03/067217
PCT Appln. WO 03/076928
PCT Appln. WO 03/087297
PCT Appln. WO 03/091426
PCT Appln. WO 03/093810
PCT Appln. WO 03/100012
PCT Appln. WO 03/100448A1
PCT Appln. WO 04/020085
PCT Appln. WO 04/027093
PCT Appln. WO 05/021570
PCT Appln. WO 05/115481
PCT Appln. WO 05/121371
PCT Appln. WO 09/923,256
PCT Appln. WO 09/936,760
PCT Appln. WO 88/10315
PCT Appln. WO 89/06700
PCT Appln. WO 90/07641
PCT Appln. WO 93/17126
PCT Appln. WO 93/23569
PCT Appln. WO 94/02595
PCT Appln. WO 94/14226
PCT Appln. WO 95/11995
PCT Appln. WO 95/21265
PCT Appln. WO 95/21944
PCT Appln. WO 95/35505
PCT Appln. WO 96/31622
PCT Appln. WO 97/10365
PCT Appln. WO 97/27317
PCT Appln. WO 97/43450
PCT Appln. WO 98/39352
PCT Appln. WO 99/14226
PCT Appln. WO 99/32619
PCT Appln. WO 99/35505
Petrocca et al., Cancer Cell, 13(3):272-286, 2008.
Pietras et al., Oncogene, 17(17):2235-2249, 1998.
Poliseno et al., Sci. Signal., 3(117):ra29, 2010.
Posner et al., Hybridoma 6, 611-625, 1987.
Prakash et al., J. Med. Chem., 48:4247-4253, 2005.
Qian et al., Biochem. Biophy. Res. Commun., 377(2):668-673, 2008.
Qin et al., Proc. Natl. Acad. Sci. USA, 95(24):14411-14416, 1998.
Rand et al., Cell, 123:621-629, 2005.
Ravindranath and Morton, Intern. Rev. Immunol., 7: 303-329, 1991.
Reichenberger et al., Cancer Res., 65(7):2668-2675, 2005.
Rosenberg et al., Ann. Surg. 210(4):474-548, 1989.
Rosenberg et al., N. Engl. J. Med., 319:1676, 1988.
Sambrook et al., In: Molecular cloning, Cold Spring Harbor Laboratory Press, Cold Spring Harbor, N.Y., 2001.
Sarver et al., Science, 247:1222-1225, 1990.
Scanlon et al., Proc. Natl. Acad. Sci. USA, 88:10591-10595, 1991.
Serrano et al., Nature, 366:704-707, 1993.
Serrano et al., Science, 267(5195):249-252, 1995.
Sharp and Zamore, Science, 287:2431-2433, 2000.
Sharp, Genes Dev., 13:139-141, 1999.
Singh et al., Chem. Commun., 4:455-456, 1998
Singh et al., J. Org. Chem., 63:10035-10039, 1998.
Soutschek et al., Nature, 432(7014):173-178, 2004.
Spitz et al., Proc. Natl. Acad. Sci. USA, 95(24):14220-14225, 1998.
Summerton and Weller, Antisense Nucleic Acid Drug Dev., 7(3):187-95, 1997.
Tabara et al., Cell, 99(2):123-132, 1999.
Taira et al., Nucleic Acids Res., 19:5125, 1991.
Thompson et al., Nature Genet., 9:444-450, 1995.
Tsujimoto and Croce, Proc. Natl. Acad. Sci. USA, 83(14):5214-5218, 1986.
Tsujimoto et al., Nature, 315:340-343, 1985.
Tyler et al. Proc. Natl. Acad. Sci. USA, 8; 96(12):7053-8, 1999.
Tyler et al., Am. J. Physiol., 277(6 Pt 1):L1199-204, 1999.
United Kingdom Appln. 8 803 000
Urban-Klein, Gene Ther., 12:461-6, 2005.
Ventura et al., Nucleic Acids Res., 21:3249, 1993.
Wahlestedt et al., Proc. Natl. Acad. Sci. USA, 97(10):5633-5638, 2000.
Walker et al., Proc. Natl. Acad. Sci. USA, 89:392-396, 1992.
Weerasinghe et al., J. Virol., 65:5531, 1991.
Wincott et al., Nucleic Acids Res., 23(14):2677-2684, 1995.
Xu et al., Curr. Biol., 13(9):790-795, 2003.
Xu et al., DNA Cell Biol., 18, 85-95, 1999.
Yan and Chen, Biochem. J., 434(1):1-10, 2011.
Yan et al., Acta Biochimica et Biophysica Sinica., 41(4):263-272, 2009.
Zavadil and Böttinger, Oncogene, 24(37):5764-5774, 2005.
Zeng et al., Cancer Res., 62(13):3630-3635, 2002.
Zhang et al., J. Biol. Chem., 275:24436-24443, 2000.
Zu et al., Cell Tissue Res., e-pub Aug. 16, 2011.

1. A method of predicting response of a cancer patient to an anti-TGF-β therapy comprising:
(a) obtaining expression information on one or more miR's from the miR 106b-25 cluster in a patient sample that contains miR's from the miR 106b-25 cluster; and
(b) classifying said subject as:
(i) anti-TGF-β-responsive if one or more miR's are observed to be upregulated as compared to normal tissue; or
(ii) anti-TGF-β-non-responsive if one or more miR's are not observed to be upregulated as compared to normal tissue.

2. The method of claim 1, further comprising making a treatment decision for said patient.

3. The method of claim 2, wherein said treatment decision is not to treat said patient with an anti-TGF-β therapy.

4. The method of claim 3, further comprising treating said patient with a therapy other than anti-TGF-β.

5. The method of claim 2, wherein said treatment decision is to treat said patient with an anti-TGF-β therapy.

6. The method of claim 5, further comprising treating said patient with an anti-TGF-β therapy.

7. The method of claim 6, wherein said anti-TGF-β therapy is an anti-TGF-β antibody.

8. The method of claim 1, further comprising measuring miR expression levels for one or more miR's that are upregulated by Six-1.

9. The method of claim 1, further comprising obtaining said sample.

10. The method of claim 9, wherein said sample is a tumor sample.

11. The method of claim 1, wherein none of miR-106b, miR-93 and miR-25 are elevated.

12. The method of claim 11, further comprising performing steps (a) and (b) a second time.

13. The method of claim 12, wherein the second time follows an anti-TGF-β therapy.

14. The method of claim 1, wherein one of miR-106b, miR-93 and miR-25 are elevated.

15. The method of claim 1, wherein two of miR-106b, miR-93 and miR-25 are elevated.

16. The method of claim 1, wherein all three of miR-106b, miR-93 and miR-25 are elevated.

17. The method of claim 14, further comprising performing steps (a) and (b) a second time.

18. The method of claim 1, wherein said cancer patient is a human patient.

19. The method of claim 1, wherein said cancer patient has breast cancer, Wilm's tumor, ovarian cancer, adenocarcinoma, adenosquamous carcinoma, papillary carcinoma, secretory carcinoma, sarcomatoid carcinoma, hepatocellular carcinoma, osteosarcoma, rhabdomyosarcoma or a peripheral nerve sheath tumor.

20. The method of claim 1, wherein said cancer is metastatic, multi-drug resistant or recurrent.

21. A method of assessing TβRI expression upregulation or increased TGF-β signaling in a cancer tissue sample comprising:
(a). obtaining expression information on one or more miR's from the miR 106b-25 cluster from said sample; and
(b). identifying TβRI upregulation or increased TGF-β signaling if one or more miR's are observed to be upregulated as compared to normal tissue.

22. (canceled)

23. A method of predicting response of a cancer patient to an anti-TGF-β therapy comprising:
(a) obtaining expression information on Six-1 in a patient sample; and
(b) classifying said subject as:
(i). anti-TGF-β-responsive if Six-1 is observed to be upregulated as compared to normal tissue; or
(ii). anti-TGF-β-non-responsive if Six-1 is not observed to be upregulated as compared to normal tissue.

24. A method of assessing TβRI expression upregulation or increased TGF-β signaling in a cancer tissue sample comprising:
(a) obtaining expression information on Six-1 from said sample; and
(b) identifying TβRI upregulation or increased TGF-β signaling if Six-1 is observed to be upregulated as compared to normal tissue.

25. (canceled)
